(19) United States
(12) Patent Application Publication
(10) Pub. No.: US 2023/0286919 A1
(43) Pub. Date: Sep. 14, 2023

(54) ISOQUINOLINE DERIVATIVES FOR USE IN THERAPY

(71) Applicants: Vestlandets Innovasjonsselskap AS, Bergen (NO); Shanghai University of Traditional Chinese Medicine, Shanghai (CN)

(72) Inventors: Yaping Hua, Bergen (NO); Karl-Henning Kalland, Bergen (NO); Weidong Zhang, Shanghai (CN); Xisong Ke, Shanghai (CN); Yunheng Shen, Shanghai (CN); Anne Margrete Øyan, Bergen (NO)

(21) Appl. No.: 17/995,143

(22) PCT Filed: Apr. 1, 2021
(86) PCT No.: PCT/EP2021/058662

§ 371 Date: Sep. 30, 2022

(30) Foreign Application Priority Data
Apr. 1, 2020 (WO) PCT/CN2020/082772
May 12, 2020 (GB) 2006986.0

(51) Int. Cl. C07D 221/16; A61P 29/00; A61K 45/06; A61P 13/08

(52) U.S. Cl. CPC C07D 221/16; A61P 29/00; A61K 45/06; A61P 13/08

(57) ABSTRACT

The present invention provides a compound of Formula (I), or a solvate, a tautomer, a stereoisomer or a salt thereof, for use in the treatment of cancer, in particular prostate cancer. These compounds are of use in cancer immunotherapy and may be used in combination therapies with immune checkpoint inhibitors and tumour microenvironment modulators. The invention also provides compositions comprising the compounds of the invention, as well as the use of compounds of the invention as STAT3 inhibitors.

### CROSS-REFERENCE TO RELATED APPLICATION(S)

The present application is a National Stage Application under 35 U.S.C. § 371 of International Application No. PCT/EP2021/058662 filed Apr. 1, 2021, which claims priority to United Kingdom Application No. 2006986.0 filed on May 12, 2020, and International Application No. PCT/CN2020/082772 filed on Apr. 1, 2020. The entire contents of these applications are incorporated herein by reference in their entireties.

### FIELD OF THE INVENTION

The present invention provides isoquinoline derivatives that are useful therapy, such as in the treatment of cancer. Also provided herein are compositions comprising the isoquinoline derivatives of the invention, as well as methods for the treatment of cancer using the isoquinoline derivatives of the invention.

### BACKGROUND OF THE INVENTION

Prostate cancer has the highest incidence of diagnosed cancer among men in Europe and the second-highest worldwide. Prostate cancer may be diagnosed based on prostate-specific antigen (PSA) screening, a digital rectal exam, multi-parametric magnetic resonance imaging (mpMRI) or gene-based tests, such as the transmembrane protease serine 2 (TMPRSS2) protein, and the non-coding RNA, prostate cancer gene 3 (PCA3).

Current treatment of prostate cancer, which includes hormone therapy, surgery, radiotherapy and chemotherapy, has slowed the progression of the disease. Androgen deprivation therapy (ADT), including drug administration and surgery, has been routine to treat prostate cancer, and patients are initially sensitive to ADT. However, in many patients, cancer relapses and progresses to a more aggressive castration-resistant prostate cancer (CRPC).

Studies show that most CRPC patients express a high level of androgen receptor (AR) and AR target genes, including prostate-specific antigen (PSA). AR mutations, constitutively active AR splice variants and activated proliferative pathways such as mitogen-activated protein kinase (MAPK) and Signal Transducer and Activator of Transcription 3 (STAT3) signalling are common in CRPC.

AR protein, which drives CRPC development, consists of several distinct functional domains: a ligand binding domain (LBD), a short hinge region, a DNA binding domain (DBD), and an amino-terminal domain (NTD). Several AR inhibitors are used clinically or are entering clinical phase studies. For example, abiraterone (Abi) blocks androgen synthesis as a CYP17A1 inhibitor, whilst seviteronel (VT-464) and galeterone (TOK-001) act as lyase inhibitors. Other AR antagonists target the AR ligand binding domain, such as enzalutamide (ENZ) and apalutamide (ARN-509). However, these AR inhibitors predominantly fail because of mutations in the LBD which create drug resistance.

The STAT3 pathway is essential to drive prostate cancer progression to metastatic CRPC. The STAT3 integrates with other signalling pathways to reactivate the AR pathway, and regulates interactions between tumor cells and the microenvironment. It has been reported that constitutively active STAT3 induced resistance to ENZ, and the JAK2 inhibitor AG490 could reverse ENZ resistance in LNCaP cells. All these observations suggest the IL-6/STAT3 pathway promotes tumorigenesis, progression and metastasis and may serve as a good target for the treatment of prostate cancer.

STAT3 was originally described in 1993 as a transcription factor from IL-6-stimulated human hepatoma (HepG2) cells. The full-length STAT3 has six different structural motifs: a transactivation domain (TAD) for co-factor recruitment; a Src Homology 2 (SH2) domain for receptor binding and dimerization; a linker domain (LD); a DNA-binding domain (DBD); a coiled-coil domain (CDD) and a conserved amino-terminal domain (NTD).

Cytokines (IL-6, IL-10, and IL-11) or growth factors (EGF, FGF, PDGF and VEGF) bind to their corresponding cell surface receptors. These bound receptors form a dimer complex, leading to the initiation and dimerization of glycoprotein 130 (gp130). A complex of the receptors and gp130 recruits Janus kinases (JAKs) and activates JAK/STAT signalling via the phosphorylation cascade. The cytoplasmic phosphorylated tyrosine residues of these receptors create a dock for the STAT3 SH2 domain. STAT3 is thereby activated through phosphorylation of tyrosine 705 (Tyr705) located in the SH2 domain.

Once activated, phosphorylated STAT3 monomers interact via their SH2 domain to form a homodimer of pSTAT3 that dissociates from the relevant receptors, translocates to the nucleus and induces gene transcription. In addition to the receptor-associated pathways like JAKs, phosphorylation of STAT3 can also be triggered by the non-receptor-associated tyrosine kinases (such as Src). DNA binding and transcriptional activity of STAT3 depend on the phosphorylation of serine (Ser 727) within the STAT3 TAD domain.

The function of STAT3 relies significantly on its SH2 domain, which promotes STAT3 homo or heterodimerization, protein-protein interactions and nuclear translocation of the STAT3 dimers needed for transcription. Thus, the STAT3 SH2 domain mediates the phosphorylation and dimerization of STAT3, due to its association between STAT3 monomers and phospho-tyrosine motifs within relevant receptors. Due to this important role, the STAT3 SH2 domain becomes a dominating therapeutic target for small molecule modulator discovery and development.

Hua et al. (Pharmacology Research & Perspectives, 2018, vol. 6, issue 6) discloses a small molecule compound (cinobufagin-3-acetate—also referred to herein as compound 154) which inhibits AR and STAT3 transcriptional activity, reduces the expression of phosphorylation of STAT3 (Y705) and downregulates the mRNA levels of AR target genes.

Another known STAT3 inhibitor is S3I-201, which has the structure shown below:

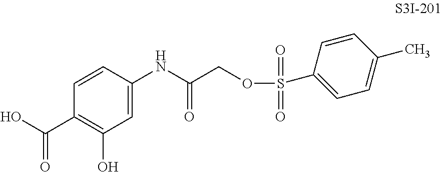 [SMILES: Cc1ccc(S(=O)(=O)OCC(=O)Nc2ccc(C(=O)O)c(O)c2)cc1]

However, is it desired to find further STAT3 inhibitors. In particular, it is desired to find compounds which can act as STAT3 SH2 domain inhibitors.

### SUMMARY OF THE INVENTION

The present invention provides compound of Formula (I), or a solvate, a tautomer, a stereoisomer or a salt thereof, for use in the treatment of (i) a STAT3-associated cancer selected from prostate, stomach, small intestine, oesophagus, melanoma, head or neck, kidney, bladder, urinary, brain, lung, pancreas, endometrium, thyroid, bile duct, gall bladder, blood vessel, appendix and rectum cancer; or (ii) an MCL-1-associated cancer selected from Chronic Lymphocytic Leukaemia and Acute Lymphocytic Leukaemia:

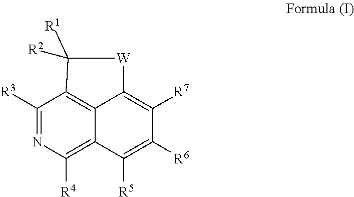

wherein
W is selected from the group consisting of —CH₂—, —CHOH—, and —C(═O)—;
R¹, R², R³, R⁴, R⁵, and R⁷ are each independently selected from the group consisting of —H, —OH, a halogen, and C₁₋₃ alkyl;
R^{x} is selected from the group consisting of C₅₋₇ cycloalkyl substituted by R^{x} and R^{y}, and C₅₋₇ cycloalkenyl substituted by R^{x} and R^{y};
R^{x} is selected from the group consisting of —OH, —NH₂, C₁₋₆ aldehyde, C₁₋₆ alcohol, C₁₋₆ alkoxy, C₂₋₆ ether, C₁₋₆ carboxylic acid, C₂₋₆ ester, C₁₋₆ amine, C₁₋₆ amide, C₂₋₆ hemiacetal, C₃₋₆ acetal, and C₃₋₄ cyclic acetal, each of which may be substituted by one or more R^{a} group;
R^{a} is selected from the group consisting of —OH, a halogen, and —NH₂; and
R^{y} is selected from the group consisting of —H, —OH, a halogen, and C₁₋₃ alkyl.

Also provided herein are compounds of the invention for use in the treatment of prostate cancer.

The present invention also provides a compound of Formula (I) or a solvate, a tautomer, a stereoisomer or a salt thereof,

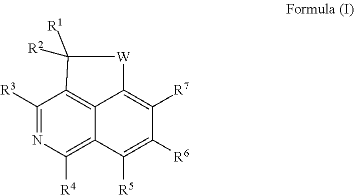

wherein
W is selected from the group consisting of —CH₂—, —CHOH—, and —C(═O)—;
R¹, R², R³, R⁴, R⁵, and R⁷ are each independently selected from the group consisting of —H, —OH, a halogen, and C₁₋₃ alkyl;
R⁶ is selected from the group consisting of C₅₋₇ cycloalkyl substituted by R^{x} and R^{y}, and C₅₋₇ cycloalkenyl substituted by R^{x} and R^{y};
R^{x} is selected from the group consisting of —OH, —NH₂, C₁₋₆ aldehyde, C₁₋₆ alcohol, C₁₋₆ alkoxy, C₂₋₆ ether, C₁₋₆ carboxylic acid, C₂₋₆ ester, C₁₋₆ amine, C₁₋₆ amide, C₂₋₆ hemiacetal, C₃₋₆ acetal, and C₃₋₄ cyclic acetal, each of which may be substituted by one or more R^{a} group;
R^{a} is selected from the group consisting of —OH, a halogen, and —NH₂; and
R^{y} is selected from the group consisting of —H, —OH, a halogen, and C₁₋₃ alkyl; with the proviso that the compound is not:

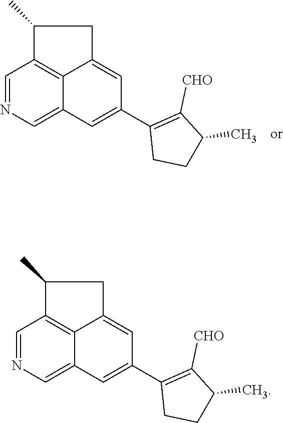 [SMILES: C[C@@H]1CCC(c2cc3c4c(cncc4c2)[C@H](C)C3)=C1C=O ; C[C@@H]1CCC(c2cc3c4c(cncc4c2)[C@@H](C)C3)=C1C=O]

Also provided herein is a composition comprising a compound of the invention and a pharmaceutically acceptable carrier.

The present invention also provides the in vitro use of a compound of the invention as a STAT3 inhibitor, preferably a STAT3 SH2 domain inhibitor.

The present invention also provides a method of treating (i) a STAT3-associated cancer selected from prostate, stomach, small intestine, oesophagus, melanoma, head or neck, kidney, bladder, urinary, brain, lung, pancreas, endometrium, thyroid, bile duct, gall bladder, blood vessel, appendix and rectum cancer; or (ii) an MCL-1-associated cancer selected from Chronic Lymphocytic Leukaemia and Acute Lymphocytic Leukaemia, wherein the method comprises administering to a patient in need thereof a therapeutically effective amount of a compound or a composition of the invention.

The present invention also provides a method of treating prostate cancer, wherein the method comprises administering to a patient in need thereof a therapeutically effective amount of a compound or a composition of the invention.

### FIGURES

FIGS. 1-4 and 6-8 show that compounds of the invention can inhibit different cell proliferations.

FIG. 5 shows the inhibitory effect of the invention on STAT3 activity.

FIGS. 9-11 and 13 show that compounds of the invention target the IL-6/STAT3 pathway.

FIG. 12 shows that compounds of the invention lower phosphorylated STAT3 at Tyr705 by targeting STAT3 directly over STAT1.

FIG. 14 shows that compounds of the invention do not affect the DNA-binding domain of STAT1, STAT3, STAT5A, STAT5B.

FIGS. 15-18 show that compounds of the invention target STAT3 directly.

FIG. 19 shows that compounds of the invention can block STAT3 dimerization by targeting the STAT3 SH2 domain.

FIGS. 20-22 show that compounds of the invention can act as direct STAT3 SH2 domain inhibitors.

FIG. 23 shows the inhibition of clonogenicity by compounds of the invention.

FIG. 24 shows flow cytometric analysis of cell apoptosis induced by treatment with compounds of the invention or cryptotanshinone for 72 hrs in DU145 cells FIGS. 25-29 show enzyme inhibitory activity of compounds of the invention on various kinases.

FIGS. 30-32 show the in silico computational docking analysis of compounds of the invention.

FIG. 33 shows that compounds of the invention can act as direct STAT3 SH2 domain inhibitors.

FIG. 34 shows a photograph of harvested LNCaP tumours after treatment.

FIG. 35 shows individual animal tumour volume and weight on the day of sacrifice.

FIG. 36 shows H&E staining of harvested representative LNCaP tumours.

FIG. 37 shows the inhibition of secreted interleukins by compounds of the invention and the comparison compound S31201.

FIG. 38 is a table reporting on inhibition of various LPS-induced dendritic markers by compounds of the invention and the comparison compounds S31201 and 154.

### DEFINITIONS

As used herein, the term “halo” refers to a halogen group, i.e. fluoro (F), chloro (Cl), bromo (Br), or iodo (I). Preferred halo or halogen groups include F, Cl and Br, with Cl being most preferred.

As used herein, the term “alkyl” refers to straight and branched saturated aliphatic hydrocarbon chains. Example alkyl groups include, but are not limited to, methyl (Me), ethyl (Et), propyl (e.g., n-propyl and isopropyl), butyl (e.g., n-butyl, isobutyl, t-butyl), and pentyl (e.g., n-pentyl, isopentyl, neopentyl).

As used herein, the term “cycloalkyl” refers to cyclized alkyl groups. The term “C₅0.7 cycloalkyl” therefore encompasses any 5-, 6- or 7-membered cyclic alkyl group. Examples of such cycloalkyl groups include, but are not limited to cyclopentyl, cyclohexyl, and norbornyl, with cyclopentyl being particularly preferred.

As used herein, the term “cycloalkenyl” refers to cyclized alkenyl groups. The term “C₅0.7 cycloalkenyl” therefore encompasses any 5-, 6- or 7-membered cyclic alkenyl group. Examples of such cycloalkenyl groups include, but are not limited to cyclopentenyl and cyclohexenyl, with cyclopentenyl being particularly preferred.

As used herein, the term “alkoxy” refers to an —O-alkyl group. The term “C₁₋₆ alkoxy” therefore encompasses any group containing an —O-alkyl group and a total of from 1 to 6 carbon atoms. Example C₁₋₆ alkoxy groups therefore include —O—(CH₂)₁₋₆—H. Other example C₁₋₆ alkoxy groups include, but are not limited to, methoxy, ethoxy, propoxy (e.g., n-propoxy and isopropoxy), and t-butoxy.

As used herein, the term “aldehyde” refers to any group containing an aldehyde (—CHO) functional group. The term “C₁₋₆ aldehyde” therefore encompasses any group containing a —CHO group and a total of from 1 to 6 carbon atoms. Example C₁₋₆ aldehyde groups therefore include —(CH₂)₀₋₅—CHO, with —CHO being particularly preferred.

As used herein, the term “alcohol” refers to any group containing an alcohol (—OH) functional group. The term “C₁₋₆ alcohol” therefore encompasses any group containing an —OH group and a total of from 1 to 6 carbon atoms. Example C₁₋₆ alcohol groups therefore include —(CH₂)₁₋₆—OH.

As used herein, the term “ether” refers to any group containing an ether (—R—O—R) functional group. The term “C₂₋₆ ether” therefore encompasses any group containing a —ROR group and a total of from 2 to 6 carbon atoms. Example C₂₋₆ ether groups include —(CH₂)ₙ—O—(CH₂)ₘ—H, wherein n and m are each from 1 to 5, provided that n+m is from 2 to 6.

As used herein, the term “carboxylic acid” refers to any group containing a carboxylic acid (—COOH) functional group. The term “C₁₋₆ carboxylic acid” therefore encompasses any group containing a —COOH group and a total of from 1 to 6 carbon atoms. Example C₁₋₆ carboxylic acid groups include —(CH₂)₀₋₅—COOH.

As used herein, the term “ester” refers to any group containing an ester (—COOR) functional group. The term “C₂₋₆ ester” therefore encompasses any group containing a —COOR group and a total of from 2 to 6 carbon atoms. Example C₂₋₆ ester groups therefore include —(CH₂)ₙ—C(═O)—O—(CH₂)ₘ—CH₃ and —(CH₂)ₙ—O—C(═O)—(CH₂)ₘ—CH₃, wherein each of n and m are from 0 to 4, provided that n+m is from 0 to 4.

As used herein, the term “amine” refers to any group containing an amine (—NR₂) functional group. The term “C₁₋₆ amine” therefore encompasses any group containing a —NR₂ group and from 1 to 6 carbon atoms. Example C₁₋₆ amine groups include —(CH₂)ₙ—N((CH₂)ₘ—H)—(CH₂)ₚ—H, wherein each of n, m and p are from 0 to 6, provided that n+m+p is from 1 to 6.

As used herein, the term “amide” refers to any group containing an amide (—CONR₂) functional group. The term “C₁₋₆ amide” therefore encompasses any group containing a —CONR₂ group and from 1 to 6 carbon atoms. Example C₁₋₆ amine groups include —(CH₂)ₙ—C(═O)—N((CH₂)ₘ—H)—(CH₂)ₚ—H, wherein each of n, m and p are from 0 to 5, provided that n+m+p is from 0 to 5.

As used herein, the term “hemiacetal” refers to any group containing an hemiacetal (—C(OH)OR) functional group. The term “C₂₋₆ hemiacetal” therefore encompasses any group containing a —C(OH)OR group and a total of from 2 to 6 carbon atoms. Example C₂₋₆ hemiacetal groups include —C(OH)—O—(CH₂)₁₋₅—H.

As used herein, the term “acetal” refers to any group containing an acetal (—C(OR)OR′) functional group. The term “C₃₋₆ acetal” therefore encompasses any group containing a —C(OR)OR′ group and a total of from 3 to 6 carbon atoms. Example C₃₋₆ acetal groups therefore include —C(O—(CH₂)ₙ—H)—O—(CH₂)ₘ—H, wherein each of n and m are from 1 to 5, provided that n+m is from 2 to 5.

As used herein, the term “cyclic acetal” refers to any acetal in which the acetal carbon and one or both oxygen atoms thereon are members of a ring. The term “C₃₋₄ cyclic acetal” encompasses any cyclic acetal containing 3 or 4 carbon atoms. Example C₃₋₄ cyclic acetal groups include dioxolane and 1,3-dioxane, with dioxolane being particularly preferred.

As used herein, the term “salts” means pharmaceutically acceptable salts. Suitable salts include organic or inorganic salts. Preferred salts include citrates, fumarates, tartarates, malates, formates, acetates, maleates, chlorides, bromides, sulphites, and sulfates.

As used herein, the term “stereoisomer” means a diastereomer or an enantiomer. More preferably, the stereoisomer is an enantiomer. The present invention generally encompasses stereoisomers of the compounds of the invention, with the exception of where specific stereochemistry is defined.

### DETAILED DESCRIPTION

The present invention provides a compound of Formula (I), or a solvate, a tautomer, a stereoisomer or a salt thereof, for use in the treatment of (i) a STAT3-associated cancer selected from prostate, stomach, small intestine, oesophagus, melanoma, head or neck, kidney, bladder, urinary, brain, lung, pancreas, endometrium, thyroid, bile duct, gall bladder, blood vessel, appendix and rectum cancer; or (ii) an MCL-1-associated cancer selected from Chronic Lymphocytic Leukaemia and Acute Lymphocytic Leukaemia:

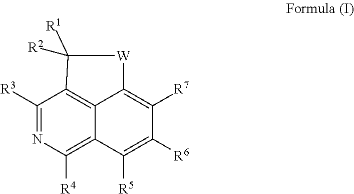

wherein
W is selected from the group consisting of —CH₂—, —CHOH—, and —C(═O)—;
R¹, R², R³, R⁴, R⁵, and R⁷ are each independently selected from the group consisting of —H, —OH, a halogen, and C₁₋₃ alkyl;
R⁶ is selected from the group consisting of C₅₋₇ cycloalkyl substituted by R^{x} and R^{y}, and C₅₋₇ cycloalkenyl substituted by R^{x} and R^{y};
R^{x} is selected from the group consisting of —OH, —NH₂, C₁₋₆ aldehyde, C₁₋₆ alcohol, C₁₋₆ alkoxy, C₂₋₆ ether, C₁₋₆ carboxylic acid, C₂₋₆ ester, C₁₋₆ amine, C₁₋₆ amide, C₂₋₆ hemiacetal, C₃₋₆ acetal, and C₃₋₄ cyclic acetal, each of which may be substituted by one or more R^{a} group;
R^{a} is selected from the group consisting of —OH, a halogen, and —NH₂; and
R^{y} is selected from the group consisting of —H, —OH, a halogen, and C₁₋₃ alkyl.

Put another way, the present invention provides a compound of Formula (I), or a solvate, a tautomer, a stereoisomer or a salt thereof, for use in the treatment of prostate cancer, stomach cancer, small intestine cancer, oesophagus cancer, melanoma, head or neck cancer, kidney cancer, bladder cancer, urinary cancer, brain cancer, lung cancer, pancreas cancer, endometrium cancer, thyroid cancer, bile duct cancer, gall bladder cancer, blood vessel cancer, appendix cancer, rectum cancer, Chronic Lymphocytic Leukaemia or Acute Lymphocytic Leukaemia.

W

W is selected from the group consisting of —CH₂—, —CHOH—, and —C(═O)—.

Preferably, W is selected from the group consisting of —CH₂—, and —C(═O)—.

More preferably, W is —CH₂—.

R¹ and R²

R¹ and R² are each independently selected from the group consisting of —H, —OH, a halogen, and C₁₋₃ alkyl.

Preferably, R¹ is —H and R² is C₁₋₃ alkyl.

More preferably, R¹ is —H and R² is Me.

R³ and R⁴

R³ and R⁴ are each independently selected from the group consisting of —H, —OH, a halogen, and C₁₋₃ alkyl.

Preferably, R³ and R⁴ are each independently selected from the group consisting of —H and a halogen.

More preferably, R³ is —H and R⁴ is a halogen, R³ is a halogen and R⁴ is —H, or R³ and R⁴ are both —H.

Most preferably, R³ and R⁴ are both H.

R⁵ and R⁷

R⁵ and R⁷ are each independently selected from the group consisting of —H, —OH, a halogen, and C₁₋₃ alkyl.

Preferably, R⁵ and R⁷ are both —H.

R⁶

R⁶ is selected from the group consisting of C₅₋₇ cycloalkyl substituted by R^{x} and R^{y}, and C₅₋₇ cycloalkenyl substituted by R^{x} and R^{y}.

Preferably, R⁶ is selected from the group consisting of C₅₋₆ cycloalkyl substituted by R^{x} and R^{y}, and C₅₋₆ cycloalkenyl substituted by R^{x} and R^{y}.

More preferably R⁶ is C₅₋₆ cycloalkenyl, which is substituted by R^{x} and R^{y}.

Even more preferably R⁶ is 1-cyclopentenyl, which is substituted by R^{x} and R^{y}.

Even more preferably, R⁶ is

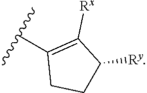

Most preferably, R⁶ is

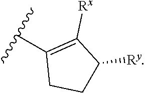

In the event that R⁶ has defined stereochemistry, the invention does not encompass stereoisomers of Formula (I). Put another way, the invention is preferably directed to compounds of Formula (Ia), or a solvate, a tautomer, or a salt thereof, for use in the treatment of (i) a STAT3-associated cancer selected from prostate, stomach, small intestine, oesophagus, melanoma, head or neck, kidney, bladder, urinary, brain, lung, pancreas, endometrium, thyroid, bile duct, gall bladder, blood vessel, appendix and rectum cancer; or (ii) an MCL-1-associated cancer selected from Chronic Lymphocytic Leukaemia and Acute Lymphocytic Leukaemia:

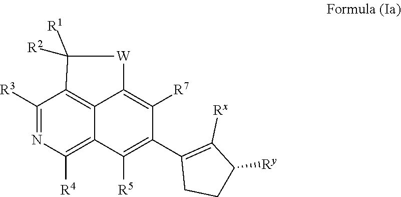

wherein
W, R¹, R², R³, R⁴, R⁵, R⁷, R^{x}, R^{a} and R^{y} are as defined herein.

R^{x}

R^{x} is selected from the group consisting of —OH, —NH₂, C₁₋₆ aldehyde, C₁₋₆ alcohol, C₁₋₆ alkoxy, C₂₋₆ ether, C₁₋₆ carboxylic acid, C₂₋₆ ester, C₁₋₆ amine, C₁₋₆ amide, C₂₋₆ hemiacetal, C₃₋₆ acetal, and C₃₋₄ cyclic acetal, each of which may be substituted (where possible) by one or more R^{a} group.

Preferably, R^{x} is substituted by at most one R^{a} group, and more preferably R^{x} is not substituted by R^{a}.

R^{x} may therefore be selected from the group consisting of —OH, —NH₂, —CHO, C₁₋₃ alkyl-CHO, C₁₋₃ alkyl-OH, —O—C₁₋₃ alkyl, —C₁₋₃ alkyl-O—C₁₋₃ alkyl, —C₁₋₃ alkyl-COOH, —C₁₋₃ alkyl-C(═O)—O—C₁₋₃ alkyl, —C₁₋₃ alkyl-O—C(═O)—C₁₋₃ alkyl, —C₁₋₃ alkyl-NH₂, —C₁₋₃ alkyl-C(═O)—NH₂, —C(OH)(—O—C₁₋₃ alkyl), —C(—O—C₁₋₃ alkyl)(—O—C₁₋₃ alkyl) and C₃₋₄ cyclic acetal, each of which may be substituted (where possible) by one or more R^{a} group.

Preferably, R^{x} is selected from the group consisting of C₁₋₆ aldehyde, C₂₋₆ hemiacetal, C₃₋₆ acetal, and C₃₋₄ cyclic acetal, each of which may be substituted by one or more R^{a} group.

R^{x} may therefore be selected from the group consisting of —CHO, C₁₋₃ alkyl-CHO, —C(OH)(—O—C₁₋₃ alkyl), —C(—O—C₁₋₃ alkyl)(—O—C₁₋₃ alkyl) and C₃₋₄ cyclic acetal, each of which may be substituted by one or more R^{a} group.

More preferably, R^{x} is selected from the group consisting of C₁₋₅ aldehyde, C₂₋₅ hemiacetal, C₃₋₅ acetal, and C₃₋₄ cyclic acetal

More preferably, R^{x} is selected from the group consisting of —CHO, —CH(OH)(OR), —CH(OR)₂, and dioxolane, wherein R is Me or Et, preferably Me.

Most preferably, R^{x} is —CHO.

R^{a}

R^{a} is selected from the group consisting of —OH, a halo group, and —NH₂.

Preferably, R^{a} is a halo group.

R^{y}

R^{y} is selected from the group consisting of —H, —OH, a halogen, and C₁₋₃ alkyl.

Preferably R^{y} is C₁₋₃ alkyl, more preferably Me.

Preferred compounds of the invention include compounds of Formula (II), and solvates, tautomers, stereoisomers and salts thereof:

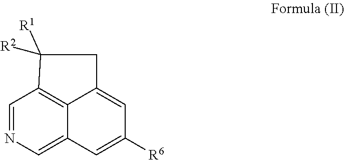

wherein R¹, R² and R⁶ are as defined above.

Preferably, R¹ is —H, R² is Me, and R⁶ is as defined above.

More preferably, R¹ is —H, R² is Me, and R⁶ is C₅ cycloalkenyl substituted by R^{x} and R^{y} or C₅ cycloalkyl substituted by R^{x} and R^{y}.

More preferred compounds of the invention include compounds of Formula (III), and solvates, tautomers, stereoisomers and salts thereof:

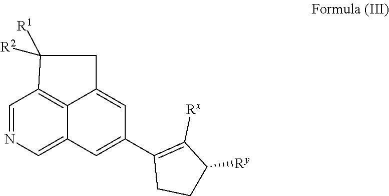

wherein R¹, R², R^{x} and R^{y} are as defined above.

Preferably, R¹ is —H, R² is Me, and R^{x} and R^{y} are as defined above.

Even more preferred compounds of the invention include compounds of Formula (IV), and solvates, tautomers, and salts thereof:

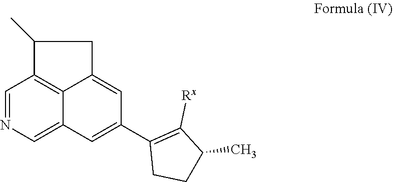

wherein R^{x} is as defined above.

Most preferred compounds for use according to the invention are the compounds 323-1 and 323-1, and salts thereof.

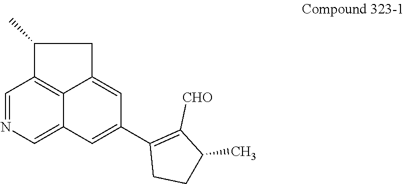 [SMILES: C[C@@H]1CCC(c2cc3c4c(cncc4c2)[C@H](C)C3)=C1C=O]

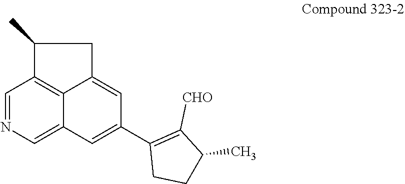 [SMILES: C[C@@H]1CCC(c2cc3c4c(cncc4c2)[C@@H](C)C3)=C1C=O]

In another aspect, the present invention provides a compound of Formula (I) or a solvate, a tautomer, a stereoisomer or a salt thereof,

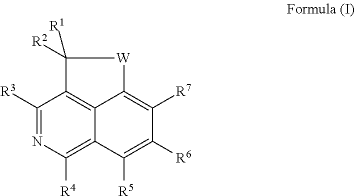

wherein
W is selected from the group consisting of —CH₂—, —CHOH—, and —C(═O)—;
R¹, R², R³, R⁴, R⁵, and R⁷ are each independently selected from the group consisting of —H, —OH, a halogen, and C₁₋₃ alkyl;
R⁶ is selected from the group consisting of C₅₋₇ cycloalkyl substituted by R^{x} and R^{y}, and C₅₋₇ cycloalkenyl substituted by R^{x} and R^{y};
R^{x} is selected from the group consisting of —OH, —NH₂, C₁₋₆ aldehyde, C₁₋₆ alcohol, C₁₋₆ alkoxy, C₂₋₆ ether, C₁₋₆ carboxylic acid, C₂₋₆ ester, C₁₋₆ amine, C₁₋₆ amide, C₂₋₆ hemiacetal, C₃₋₆ acetal, and C₃₋₄ cyclic acetal, each of which may be substituted by one or more R^{a} group;
R^{a} is selected from the group consisting of —OH, a halogen, and —NH₂; and
R^{y} is selected from the group consisting of —H, —OH, a halogen, and C₁₋₃ alkyl; with the proviso that the compound is not:

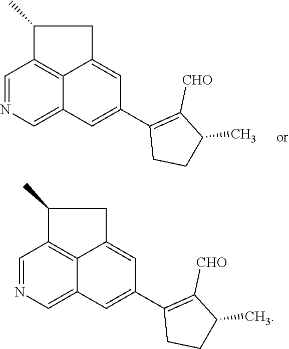 [SMILES: C[C@@H]1CCC(c2cc3c4c(cncc4c2)[C@H](C)C3)=C1C=O ; C[C@@H]1CCC(c2cc3c4c(cncc4c2)[C@@H](C)C3)=C1C=O]

All of the preferred definitions for the groups W, R¹, R², R³, R⁴, R⁵, R⁶, R⁷, R^{x}, R^{a} and R^{y} in this aspect of the invention are the same as those set out above.

Thus, in one aspect the present invention provides a compound of Formula (Ia) or a solvate, a tautomer, or a salt thereof, wherein Formula (Ia) is as defined above, with the proviso that the compound is not:

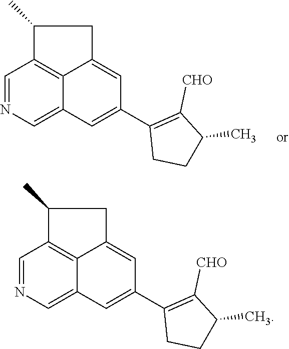 [SMILES: C[C@@H]1CCC(c2cc3c4c(cncc4c2)[C@H](C)C3)=C1C=O ; C[C@@H]1CCC(c2cc3c4c(cncc4c2)[C@@H](C)C3)=C1C=O]

In another aspect the present invention provides a compound of Formula (II) or a solvate, a tautomer, a stereoisomer or a salt thereof, wherein Formula (II) is as defined above, with the proviso that the compound is not:

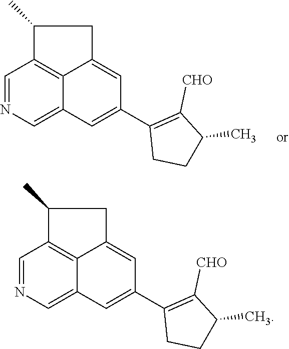 [SMILES: C[C@@H]1CCC(c2cc3c4c(cncc4c2)[C@H](C)C3)=C1C=O ; C[C@@H]1CCC(c2cc3c4c(cncc4c2)[C@@H](C)C3)=C1C=O]

In another aspect the present invention provides a compound of Formula (III) or a solvate, a tautomer, a stereoisomer or a salt thereof, wherein Formula (III) is as defined above, with the proviso that the compound is not:

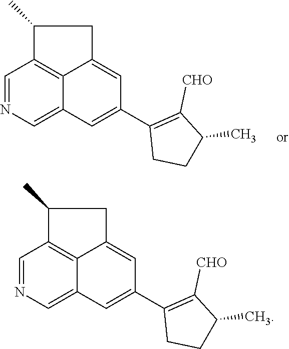 [SMILES: C[C@@H]1CCC(c2cc3c4c(cncc4c2)[C@H](C)C3)=C1C=O ; C[C@@H]1CCC(c2cc3c4c(cncc4c2)[C@@H](C)C3)=C1C=O]

In another aspect the present invention provides a compound of Formula (IV) or a solvate, a tautomer, or a salt thereof, wherein Formula (IV) is as defined above, with the proviso that the compound is not:

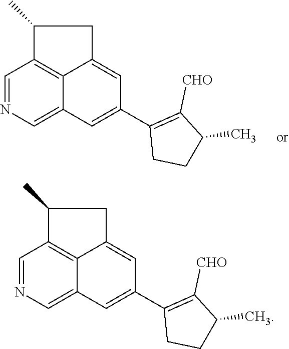 [SMILES: C[C@@H]1CCC(c2cc3c4c(cncc4c2)[C@H](C)C3)=C1C=O ; C[C@@H]1CCC(c2cc3c4c(cncc4c2)[C@@H](C)C3)=C1C=O]

The compounds of the invention may be made using any suitable synthetic process, and the skilled person would be aware of suitable synthetic routes for the compounds disclosed herein. For example, the total synthesis of compounds 323-1 and 323-2 has been reported by Zhang et al. (Journal of the American Chemical Society. 2017; 139: 5558-67). In addition, compounds 323-1 and 323-2 are natural products and may therefore be isolated from natural sources. Other compounds of the invention may be synthesized starting from compounds 323-1 and 323-2, as described in the Examples. Alternatively, the synthesis of compounds 323-1 and 323-2 could be adjusted to produce other compounds of the invention. The skilled person would be well aware of suitable adjustments that could be made to the total synthesis of compounds 323-1 and 323-2 (such as that suggested by Zhang et al.) in order to formulate other compounds of the invention.

The compounds of the present invention (e.g. compounds of Formula (I), (II), (Ill) or (IV)), can act as STAT3 SH2 domain inhibitors. Without wishing to be bound by theory, it is believed that the compounds of the invention can act as STAT3 inhibitors by targeting the STAT3 SH2 domain and inhibiting both phosphorylated and unphosphorylated STAT3 dimerization. The compounds are believed to directly target STAT3 over STAT1, without affecting SRC or JAK2. The compounds of the invention therefore have the potential to inhibit tumor cell proliferation.

The present invention therefore provides a compound of the invention (e.g. a compound of Formula (I), (II), (Ill) or (IV)) or a solvate, stereoisomer, tautomer, or salt thereof, for use in the treatment of (i) a STAT3-associated cancer selected from prostate, stomach, small intestine, oesophagus, melanoma, head or neck, kidney, bladder, urinary, brain, lung, pancreas, endometrium, thyroid, bile duct, gall bladder, blood vessel, appendix and rectum cancer; or (ii) an MCL-1-associated cancer selected from Chronic Lymphocytic Leukaemia and Acute Lymphocytic Leukaemia.

The cancer to be treated is preferably a STAT3-associated cancer, i.e. a cancer which involves one or more STAT3 mutations. An induced myeloid leukaemia cell differentiation protein (MCL-1)-associated cancer is a cancer associated with this protein, for example associated with aberrant MCL-1 expression, signaling, behavior or activity.

The cancer to be treated is therefore preferably stomach, small intestine, oesophagus, melanoma, head or neck (e.g. upper aerodigestive tract or salivary gland), prostate, kidney, bladder, urinary, brain, lung, pancreas, endometrium, thyroid, bile duct, gall bladder, blood vessel, appendix, or rectum cancer.

The cancer to be treated is more preferably prostate cancer, even more preferably castration resistant prostate cancer (CRPC), including metastatic CRPC. Preferred patients to be treated are those exhibiting constitutive activation of the STAT3 signaling pathway. Alternatively viewed, preferred tumours to be treated according to the present invention are those which are driven by STAT3, e.g. in which the STAT3 signaling pathway is upregulated.

Preferred prostate cancer to be treated is metastatic prostate cancer, i.e. cancer which has resulted in secondary tumours which are located in regions of the body outside the prostatic capsule. The primary and/or secondary tumours may be targets for treatment in this scenario. In general, the present invention is particularly suited for treatment of advanced prostate cancer, including CRPC. Advanced prostate cancer includes prostate cancer with a Gleason (biopsy) score of 8-10.

The present invention also provides a composition comprising a compound of the invention (e.g. a compound of Formula (I), (II), (Ill) or (IV)) or a solvate, stereoisomer, tautomer, or salt thereof, and a pharmaceutically acceptable carrier.

The present invention also provides a composition as defined above, for use in the treatment of (i) a STAT3-associated cancer selected from prostate, stomach, small intestine, oesophagus, melanoma, head or neck, kidney, bladder, urinary, brain, lung, pancreas, endometrium, thyroid, bile duct, gall bladder, blood vessel, appendix and rectum cancer; or (ii) an MCL-1-associated cancer selected from Chronic Lymphocytic Leukaemia and Acute Lymphocytic Leukaemia. Preferred cancers are as discussed above.

The present invention also provides a composition as defined above, for use in the treatment of prostate cancer.

As used herein, the term “treatment” includes prophylactic treatment. Treatment includes reducing the tumour mass, reducing the growth of tumour mass or reducing the formation of metastases.

The present invention also provides the in vitro use of a compound of the invention (e.g. a compound of Formula (I), (II), (III) or (IV)) or a solvate, stereoisomer, tautomer, or salt thereof, as a STAT3 inhibitor, preferably a STAT3 SH2 domain inhibitor.

The present invention also provides the use of a compound of the invention (e.g. a compound of Formula (I), (II), (Ill) or (IV)) or a solvate, stereoisomer, tautomer, or salt thereof in the manufacture of a medicament for use in the treatment of (i) a STAT3-associated cancer selected from prostate, stomach, small intestine, oesophagus, melanoma, head or neck, kidney, bladder, urinary, brain, lung, pancreas, endometrium, thyroid, bile duct, gall bladder, blood vessel, appendix and rectum cancer; or (ii) an MCL-1-associated cancer selected from Chronic Lymphocytic Leukaemia and Acute Lymphocytic Leukaemia.

The present invention also provides the use of a compound of the invention (e.g. a compound of Formula (I), (II), (Ill) or (IV)) or a solvate, stereoisomer, tautomer, or salt thereof in the manufacture of a medicament for use in the treatment of prostate cancer.

The present invention also provides a method of treating (i) a STAT3-associated cancer selected from prostate, stomach, small intestine, oesophagus, melanoma, head or neck, kidney, bladder, urinary, brain, lung, pancreas, endometrium, thyroid, bile duct, gall bladder, blood vessel, appendix and rectum cancer; or (ii) an MCL-1-associated cancer selected from Chronic Lymphocytic Leukaemia and Acute Lymphocytic Leukaemia (preferred cancers and tumours to be treated are as described elsewhere herein), wherein the method comprises administering to a patient in need thereof a therapeutically effective amount of a compound or composition of the invention, as defined herein.

The present invention also provides a method of treating prostate cancer (preferred prostate cancers to be treated are as described elsewhere herein), wherein the method comprises administering to a patient in need thereof a therapeutically effective amount of a compound or composition of the invention, as defined herein.

The present invention also provides a method of inhibiting STAT3 or the STAT3 pathway in a patient (and thereby treating cancer in the patient), wherein the method comprises administering to a patient in need thereof a therapeutically effective amount of a compound or composition of the invention. Preferred cancers and compounds are as discussed above. A therapeutically effective amount will be determined based on the clinical assessment and can be readily monitored.

As well as a direct cytotoxic effect on cancer cells, the present inventors have shown that the compounds the subject of the present invention have utility in the treatment of cancer by immunotherapy. In immunotherapy, one aim is to stimulate selected immune cells, such as antigen-presenting dendritic cells (DCs), and this may be achieved by inhibiting STAT3 signal transduction that leads to modulation of pro-inflammatory and anti-inflammatory features. It is known that STAT3 inhibition may promote pro-inflammatory features of dendritic cells. This is a desirable effect in immunotherapy. Compounds that can inhibit and/or be cytotoxic to cancer cells and also be stimulating to relevant immune cells are highly desirable. As reported in the Examples, compounds of the invention inhibit pSTAT3, reduce LPS-induced IL-10 and IL-12 production, inhibit DCs maturation (e.g. as shown by CD83) and suppress the production of PD-L1 and PD-L2 in lipopolysaccharide (LPS)-induced mature DCs. Thus these compounds represent promising new cancer immunotherapeutics.

Current immune checkpoint (ICP)-targeting agents such as PD-L1 inhibitors do not perform well against so called “cold” tumours (non-immunogenic tumours that have not yet been infiltrated with T cells). Thus, there are limitations with this promising recent class of anti-cancer agent and there remains a need for additional immuno-modulators to enhance the efficiency of cancer treatment.

In addition, the tumour microenvironment (TME) affects the efficacy of immuno-cancer treatments due to intrinsic tumour and local adaptive immunosuppression. Intrinsic tumour immunosuppression involves genetic alterations and different oncogenic pathways, such as WNT-β-catenin, mitogen-activated protein kinase (MAPK), Janus kinase (JAK)-signal transducer and activation of STAT3 and nuclear factor-KB (NF-KB) signalling pathways. These signalling pathways associate with abnormal expression of cytokines and chemokines that largely determine the exclusion of T cells (“cold” immune response) or suppression of T cell recruitment (“altered” immune response). Thus it would be advantageous to use the STAT3 inhibitors described herein to modulate the TME, especially in combination with other ICP targeting agents or TME modulators.

Thus, in a further aspect, the present invention provides a compound of the invention (e.g. a compound of Formula (I), (II), (III) or (IV)) or a solvate, stereoisomer, tautomer, or salt thereof for use in cancer immunotherapy.

In a further aspect, the present invention provides a compound of the invention (e.g. a compound of Formula (I), (II), (III) or (IV)) or a solvate, stereoisomer, tautomer, or salt thereof for use in cancer immunotherapy wherein the compound is used in combination with (i.e. administered as part of a therapeutic regimen with) an immune checkpoint inhibitor and/or a tumour microenvironment modulator.

Alternatively viewed, the present invention provides a compound of the invention (e.g. a compound of Formula (I), (II), (Ill) or (IV)) or a solvate, stereoisomer, tautomer, or salt thereof for use in cancer immunotherapy by combined, sequential or separate administration with an immune checkpoint inhibitor and/or a tumour microenvironment (TME) modulator.

Preferred cancer targets are discussed above and prostate cancer and more particularly CPRC are especially preferred. Further preferred cancer targets include head and neck squamous cell carcinoma, non-small cell lung cancer (NSCLC, squamous and non-squamous carcinoma), melanoma, urothelial and kidney cancers, Merkel cell carcinoma, refractory Hodgkin lymphoma, microsatellite instability-high colorectal cancer and gastric cancer.

Preferred cancer targets are “cold” tumours.

In a further aspect the present invention provides a pharmaceutical pack or pharmaceutical composition comprising:
(i) a compound of the invention; and
(ii) an immune checkpoint inhibitor and/or a tumour microenvironment (TME) modulator.

Immune checkpoints are well known in the art and include CTLA4, PD-1, PD-L1 and PD-L2, these are also preferred according to the present invention. Checkpoint inhibitors are also well known in the art and include antibodies against the immune checkpoints (e.g. antibodies against CTLA4, PD-1, PD-L1 and PD-L2), such as ipilimumab, nivolumab, pembrolizumab and durvalumab. Antibodies are preferred immune checkpoint inhibitors.

The significance of the TME has also gained greater recognition in recent years and become a target for therapeutic intervention (Nguyen et al, Journal of Cell Biology (2020) 219 (1): e201908224 and Tang et al., Signal Transduction and Targeted Therapy volume 6, Article number: 72 (2021)) and TME modulators are known in the art. Aspects which may be targeted include antigen presenting capability, immune cell infiltration and tumour infiltration. Molecular targets include MAPK, JAK, STAT3, beta-catenin, IDO and OX40 and these are preferred TME targets according to the present invention. So far as specific drugs are concerned, metronomic (low dose) cyclophosphamide or an anti-OX40 antibody may be preferred.

One particularly preferred combination according to the invention is cyclophosphamide (low/metronomic dose) and/or an antibody against one or more of CTLA4, PD-1, PD-L1 and PD-L2 together with a compound of the present invention.

Subjects or patients treated in accordance with the present invention will preferably be humans but veterinary treatments are also contemplated.

The compositions (formulations), e.g. pharmaceutical compositions, according to the invention may be presented, for example, in a form suitable for oral, nasal, parenteral, intravenal, topical, intratumoural or rectal administration. As used herein, the term “pharmaceutical” includes veterinary applications of the invention.

The active compounds defined herein (e.g. the compounds of Formula (I), (II), (Ill) or (IV)) may be presented in the conventional pharmacological forms of administration, such as tablets, coated tablets, nasal sprays, solutions, emulsions, liposomes, powders, capsules or sustained release forms.

Tablets may be produced, for example, by mixing the active ingredient or ingredients with known excipients, such as for example with diluents, such as calcium carbonate, calcium phosphate or lactose, disintegrants such as corn starch or alginic acid, binders such as starch or gelatin, lubricants such as magnesium stearate or talcum, and/or agents for obtaining sustained release, such as carboxypolymethylene, carboxymethyl cellulose, cellulose acetate phthalate, or polyvinylacetate.

The tablets may if desired consist of several layers. Coated tablets may be produced by coating cores, obtained in a similar manner to the tablets, with agents commonly used for tablet coatings, for example, polyvinyl pyrrolidone or shellac, gum arabic, talcum, titanium dioxide or sugar. In order to obtain sustained release or to avoid incompatibilities, the core may consist of several layers too. The tablet-coat may also consist of several layers in order to obtain sustained release, in which case the excipients mentioned above for tablets may be used.

Organ specific carrier systems may also be used.

Injection solutions may, for example, be produced in the conventional manner, such as by the addition of preservation agents, such as p-hydroxybenzoates, or stabilizers, such as EDTA. The solutions are then filled into injection vials or ampoules.

Nasal sprays may be formulated similarly in aqueous solution and packed into spray containers either with an aerosol propellant or provided with means for manual compression. Capsules containing one or several active ingredients may be produced, for example, by mixing the active ingredients with inert carriers, such as lactose or sorbitol, and filling the mixture into gelatine capsules.

Suitable suppositories may, for example, be produced by mixing the active ingredient or active ingredient combinations with the conventional carriers envisaged for this purpose, such as natural fats or polyethyleneglycol or derivatives thereof.

The compounds of the invention are preferably administered by injection, more preferably intraperitoneal injection.

Suitable dosages for the compounds of the invention are preferably in the amount of from 5 to 100 mg/kg, more preferably from 25 to 50 mg/kg, for intraperitoneal injection in mice. The skilled person would be well aware of how to adjust these dosages for other modes of administration and/or for administration to other subjects (such as humans).

The invention will now be described by way of the following non-limiting Examples.

### Example 1—Demonstration of Efficacy

Materials and Methods

Cell Culture and Reagents

Human prostate cancer cell lines LNCaP and 22Rv1 were purchased from the American Type Culture Collection (ATCC) and cultured in RPMI 1640 medium with 10% FCS. 293 and DU145 cells (ATCC) were cultured in DMEM medium with 10% FCS. EPT3-M1-STAT3 were cultured in Ham's F-12 medium with 10% FCS. Cryptotanshinone, IL-6 were purchased from (Sigma Aldrich, St. Louis, Mich., USA). S3I-201 was bought from Thermo Fisher Scientific (Waltham, Mass., USA).

MTS Assay

PC3, 22Rv1, LNCaP, EPT3-M1-STAT3 cells were seeded in 96-well plates for 24 hrs followed by treatment with different doses of 323-1 or 323-2. MTS assay was done after 3 days by adding 10 μl/well CellTiter 96@ AQueous One Solution Reagent (Promega, Madison, Wis., USA) for 4 hrs and the absorbance at 490 nm recorded using a Biotek machine.

Plasmids and Transfection

HEK 293T cells were transiently transfected with Cignal STAT3 reporter (SABioscience, Qiagen, Venlo, Netherlands) using Lipofectamine 3000 transfection reagent (Thermo Fisher Scientific, Waltham, Mass., USA) for 24 hours, following treatment with 20 ng/ml IL-6 and the indicated concentrations of 323-1, 323-2, S3I-201 or Cryptotanshinone. Luciferase activity was measured by the Dual Luciferase Assay Kit (Promega) using a Luminescence Microplate Reader (Biotek). Values were normalized to Renilla luciferase activity of DMSO vehicle.

DARTS Assay

EPT3M1-STAT3 cells were lysed with cold M-PER buffer (Thermo Fisher Scientific, Pierce cat.78501) containing protease (Roche, Basel, Switzerland, cat. no. 11836153001) and phosphatase inhibitors (Pierce cat. no. 78420) and centrifugated (18,000×g for 10 min at 4° C.). Lysates were diluted to the same final volume and proteolysed in TNC buffer [50 mM Tris-HCl (pH 8.0), 50 mM NaCl, 10 mM CaCl2]. 200 μM 323-1 or 200 μM 323-2 or the same volume of DMSO were added and incubated for 1 hour at RT. Pronase solution (1.25 mg/ml, 1:100 stock solution) was diluted serially by mixing with 1×TNC buffer to create 1:300, 1:1000, 1:3000 and 1:10000 Pronase stock aliquots. Pronase was added into both DMSO and drug groups and incubated for 30 min at RT. Digestion was stopped by adding 4× loading buffer and heating to 90° C. for 10 min immediately prior to the Western blot assay according to Lomenick, et al. (Proc Natl Acad Sci USA, 2009; 106: 21984-9) and Lomenick wt al. (ACS Chem Biol. 2011; 6: 34-46).

Immunofluorescence Staining

EPT3M1-STAT3 cells were treated with DMSO, 20 μM 323-1 or 323-2, 100 μM S3I-201 or 5 μM cryptotanshinone for 24 hrs. Cells were fixed with 4% paraformaldehyde, and mounted onto Millipore microscope slides with 7 μl of ProLong® Gold Antifade Mountant (Thermo Fisher Science) with DAPI. Images were examined using the LeicaDMRBE microscope or Cytation5 Cell Imaging Multi-Mode Reader.

SelectScreen™ Biochemical Kinase Profiling

The in vitro kinase inhibitory assays in the presence of compounds 323-1 and 323-2 were carried out according to the Z′-LYTE™ Screening and LanthaScreen Eu Kinase Binding Assays Experimental Procedures in Invitrogen (ThermoFisher Scientific, Waltham, Mass., USA). For the Z′-LYTE™ Screening profiling for FRAP1 (mTOR), JAK3, and LTK (TYK1), a mixture containing the test compounds in 100% DMSO, 2.4 μL Kinase buffer (50 mM HEPES pH 7.5, 0.01% BRIJ-35, 10 mM MgCl2, 1 mM EGTA), 5 μL 2× Peptide/Kinase Mixture, 2.5 μL 4×ATP Solution were added into black 384-well low volume NBS and bar-coded corning plate (Corning Cat. #4514) and shake the plate for 30 seconds. After incubation at RT for 60 min, 5 μL Development Reagent Solution was added into plate and shake the plate for 30 seconds, followed by Development Reaction incubation at RT for 60 min. Read on fluorescence plate reader and analyze the data. For the LanthaScreen Eu Kinase Binding Assays of MAP2K4 (MEK4) and TGFBR1 (ALK5) the previous mixture is composed of 160 nl of compounds in 100% DMSO, 3.84 μL Kinase buffer, 8 μL 2× Antibody/Kinase Mixture, and 4 μL 4× Tracer.

DNA Binding Assay

DU145 cells were seeded and treated with 5-20 μM 323-1 or 323-2, or 100 μM S3I-201 for 24 hrs. The nuclear extraction was performed according to the STAT Family Transcription Factor Assay Kit (Abcam, ab207228). First, cells were washed with ice-cold PBS and Phosphatase Inhibitors Cocktail (125 mM NaF, 250 M p-glycerophosphatase, 250 mM PNPP, 25 mM NaVO3) in ice-cold PBVS was added and cells scraped off the dish. Then cells were collected at 300×g for 5 minutes at 4° C. The pellet was resuspended in hypotonic buffer (20 mM Hepes pH7.5, 5 mM NaF, 10 μM Na2MoO4, 0.1 mM EDTA) and 10% NP-40 was added to a final concentration 0.5% and mixed well. The mixture was centrifugated at 4° C. for 30 seconds in a microcentrifuge and supernatant was discarded. The nuclear pellet was resuspended in Complete Lysis Buffer (containing 1 M DTT and Protease Inhibitor Cocktail Cocktail on ice for 30 minutes on a shaking platform followed by centrifugation at 14,000×g for 10 minutes at 4° C. to collect the supernatant as the nuclear extract. Extracted proteins were added to the pre-coated 96-well plate with an oligonucleotide containing the STAT consensus binding site. Alternatively, the Nb2 nuclear extract (Prolactin stimulated) was added together with wild-type oligonucleotide or mutated oligonucleotide to the pre-coated 96-well plate with an oligonucleotide containing the STAT consensus binding site. Plates were incubated at RT for 1 hour, followed by addition of the relevant primary antibody (STAT1, STAT3, STAT5a or STAT5b) to wells and incubation for 1 hr at RT. After washing the wells, HRP-conjugated secondary antibody was added to the wells and incubated for 1 hour at RT. Wells were washed before developing solution was added. Stop solution was added when the wells turned medium to dark blue followed by measurement of absorbance at OD 450 nm.

Western Blot Analysis

The levels of expression of phosphorylated STAT3 (Y705), STAT1, MCL1, pSTAT1 (Y701), pSTAT1 (S727) and STAT3 proteins were determined by Western blotting following the procedures described by Liu et al. (Chem Biol. 2014; 21: 1341-50). The following antibodies from Abcam, Cambridge, UK, were used in Western blotting: anti-pSTAT3 (Tyr705) (ab76315, 1/5000); anti-total STAT3 (ab119352, 1/2500); anti-pSTAT1 (Y701) (ab30645, 1/500); anti-pSTAT1 (S727) (ab86132, 1/500); anti-total STAT1 (ab47425, 1/500); anti-JAK2 (ab108596, 1/1000); anti-SRC (ab47405, 1/500); anti-PARP (G7341, 1/1000); anti-MCL1 (ab32087, 1/1000); anti-Cyclin-D1 (ab10540, 1/500); anti-BCL-XL (ab32370, 1/500); anti-survivin (ab76424, 1/2000); anti-p-actin (ab8226, ab8227, 1/2000); anti-GAPDH (ab181602, 1/2500); anti-HA (ab9110, 1/5000); anti-FLAG (DDDDK tag) (ab1162, 1/10000).

Co-Immunoprecipitation

7.5×10⁶ 293T cells were transfected with 10 μg/10 cm dish FLAG-STAT3 and 10 μg/10 cm dish HA-STAT3 plasmids for 24 hrs, followed by 20 μM 323-1 or 323-2, 100 μM S3I-201 for 4 or 24 hours. 293T cells were collected in the medium and centrifuged at 800 g x 5 min, then washed with cold PBS twice before adding 500 μl Pierce™ IP Lysis Buffer (Thermofisher Scientific, 87787), containing protease and phosphatase inhibitor cocktail without DTT (Roche, 11836153001), into the cells. Cell lysates were passed several times through a 27^{1/2}-gauge needle to disrupt the nuclei. 1-2 mg extracts were added with pre-cleaned 50 μL slurry of Pierce™ DYKDDDDK Magnetic Agarose (Thermoscientific, A36797), then supernatant were collect with magnet and immunoprecipitated (IP) at RT on a rotator for 30 min. Beads were boiled at 95° C. for 5 min followed by adding the 100 μL of 1× Non-reducing Sample Buffer (Thermofisher Scientific, 39000) into beads. The supernatant was collected with a magnet and then added 5 μL of 1M DTT (Thermoscientific, P2325) into sample prior to proceeding the immunoblotting.

Computational Docking of STAT3

The computational docking of compounds 323-1 and 323-2 to three-dimensional crystal structures of STAT3, including the phosphorylated (PDB entry 1BG1) and the nonphosphorylated (PDB entry 3CWG) structure, was utilized by the molecular docking tool Maestro 9.0 Glide as illustrated by Zheng et al. (Mycobacterium smegmatis. FEBS Letters. 2018; 592: 1445-57). The low-energy conformers of compounds 323-1 and 323-2 was generated by the Ligprep module of maestro, while the protein models were established via removing crystallized solvent molecules, reassigning the bond order, and supplement of hydrogen atoms. The minimization of energy in an OPLS-2005 force field was applied with an rmsd less than 18 Å and the number of conformations was set to 100. The docking simulation applied the three pockets of STAT3 sH2 domain for the binding site. The calculation of docking score (kcal/mol) was conducted to verify the capacity of protein-ligand interactions.

Fluorescence Polarization (FP) Assay

The FP assay was conducted as described by Zhao et al. (J Biol Chem. 2010; 285: 35855-65). The labeled phosphopeptide, 5-FLU-G(P-TYR)LPQTV-NH2 was synthesized by ProImmune (London, UK) with over 95% purity as probe. The Recombinant Human STAT3 protein was purchased from Abcam (ab43618). For saturation curves, various concentration of peptide probe (0-100 nM) and STAT3 protein (0-1000 μM) were incubated for 30 min at RT in the buffer (50 mm NaCl, 10 mm HEPES, 1 mm EDTA, 0.1% Nonidet P-40). To obtain the inhibitory effect, drugs (0-1000 μM) and 150 nM human STAT3 were incubated at 37° C. for 1 hour prior to co-incubation with 10 nM of labeled LPQTV peptide at RT for 30 min. The plate was sealed under nitrogen and left at RT for 72 hours. The fluorescence polarization was then analyzed by the BioTek Synergy™ Neo2 Multi-Mode Microplate Reader (BioTek Instruments, Winooski, Vt., USA). Ki=IC50/(([L])/Kd)+1).

Antitumor Activity In Vivo

Four-week-old male BALB/c-nu nude mice (15-20 g) were obtained from the Shanghai BiKai Laboratory Animal Co., Ltd. (Shanghai, China). The animals were maintained under specific pathogen-free conditions with food and water supplied ad libitum in the Laboratory Animal Center of the Second Military Medical University. All animal experiments were carried out in accordance with the Guide for the Care and Use of Laboratory Animals of the National Institutes of Health and were approved by the Committee on the Ethics of Animal Experiments of the Second Military Medical University, China. LNCaP cells were harvested and re-suspended in PBS. A total of 5×10⁶ cells were subcutaneously injected into the right flank. Tumour volume was calculated using the following formula: V=(L×WP)/2, where L is the length and W is the width of the tumour nodules measured with vernier callipers. Once the volume of the tumours reached 75-100 mm³, the mice were randomly divided into three groups (n=6). The mice were treated daily for three weeks with IP injections of either vehicle (olive oil) or compound 323-2 (either 20 mg/kg or 40 mg/kg in the vehicle). Body weights and tumour volumes were measured before each drug injection. After the 22nd day, the mice were euthanized, and the tumours were isolated, weighed and photographed.

Statistics

Significance in groups was determined by Student's t test or SAM (statistical analysis of microarrays) for microarray data and P value<0.05 was considered significant.

Results

Compounds 323-1 and 323-2 are Modulators of the STAT3 Over STAT1 Pathway

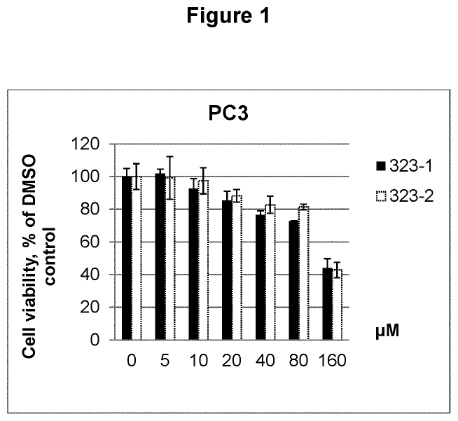

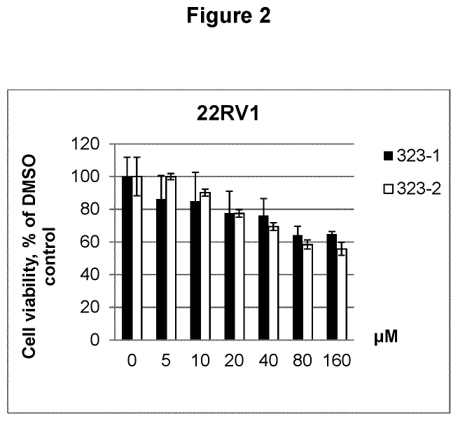

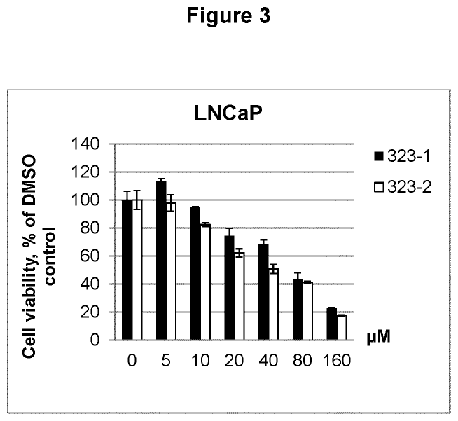

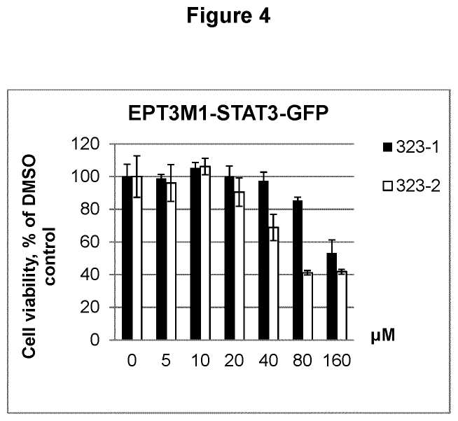

In a screen of nearly 600 natural compounds, (15R,2R)-Delavatine A (compound 323-1) and (15S,2R)-Delavatine A (compound 323-2) were found to efficiently inhibit the proliferation of human prostate tumor-initiating cells (TICs) and other tumor cells in a dose-dependent manner after treatment for 24 hours. The cells tested were PC3, 22Rv1, LNCaP, EPT3-M1-STAT3-GFP, and the results are shown in FIGS. 1-4.

To obtain the results shown in FIGS. 1-4, different cell lines were treated with indicated doses of compounds 323-1 and 323-2 for 72 hrs. All data are represented as the average±s.e.m (n=3). Significance was verified by using unpaired two-tailed Student's t-test. *p s 0.05 The total synthesis of compounds 323-1 and 323-2 was conducted as previously reported by Zhang et al. (Journal of the American Chemical Society. 2017; 139: 5558-67). The chemical structures of compounds 323-1 and 323-2 were validated by ¹H NMR, ¹³C NMR.

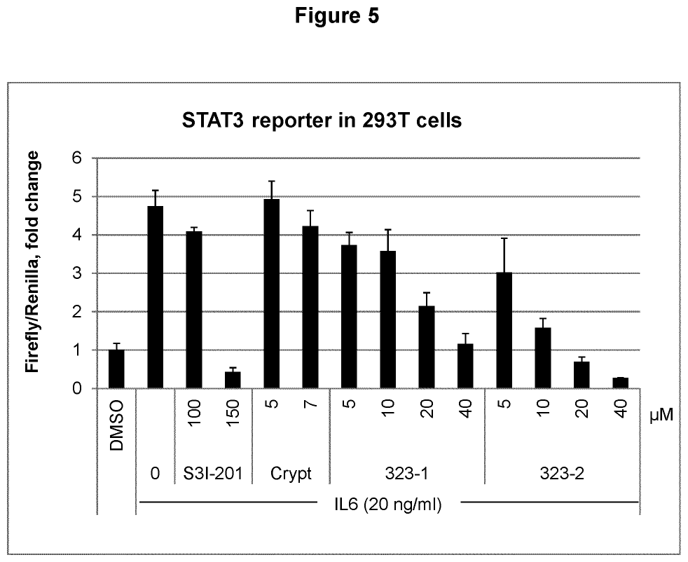

To confirm the inhibitory effect of compounds 323-1 and 323-2 on STAT3 activity, luciferase assay was carried out using a STAT3-luciferase reporter vector in 293T cells, following treatment with 323-1, 323-2 or the commercial STAT3 inhibitors S3I-201 and cryptotanshinone in the presence of IL-6 for 24 hours. The results are shown in FIG. 5.

S3I-201 shows potent inhibition of STAT3 DNA binding activity with IC-50 of 86 μM in cell-free assays, and low activity towards STAT1 and STAT5 and with STAT3 inhibition of cell lines typically at concentrations from 50 to 100 μM.

Cryptotanshinone is a STAT3 inhibitor with IC50 of 4.6 μM in a cell-free assay, strongly inhibits phosphorylation of STAT3 Tyr705, but none against STAT1 nor STAT5.

Cryptotanshinone is reported to inhibit STAT3 of cell lines typically at concentrations from 5 to 50 μM.

As shown in FIG. 5, compounds 323-1 and 323-2 exhibited a stronger inhibitory effect on STAT3 activation than cryptotanshinone

To obtain the results shown in FIG. 5, HEK293T cells transfected with STAT3 luciferase reporter were treated with different doses of compound 323-1 or compound 323-2, S3I-201 or cryptotanshinone (Crypt) in the presence of 20 ng/ml IL-6 for 24 hrs. All data are represented as the average±s.e.m (n=3). Significance was verified by using unpaired two-tailed Student's t-test. *p s 0.05.

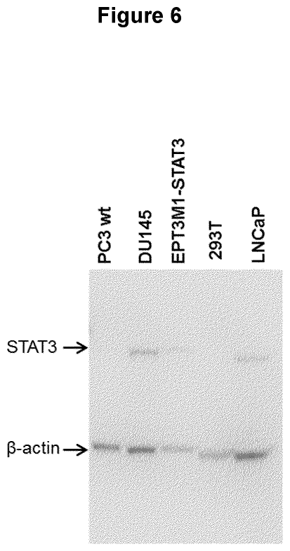

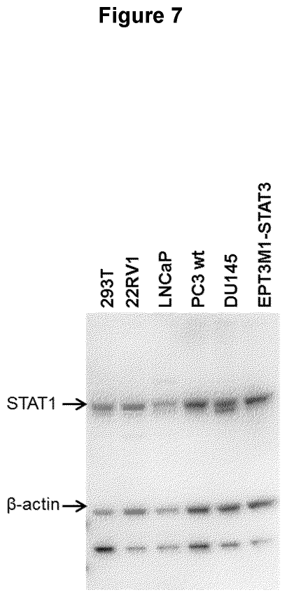

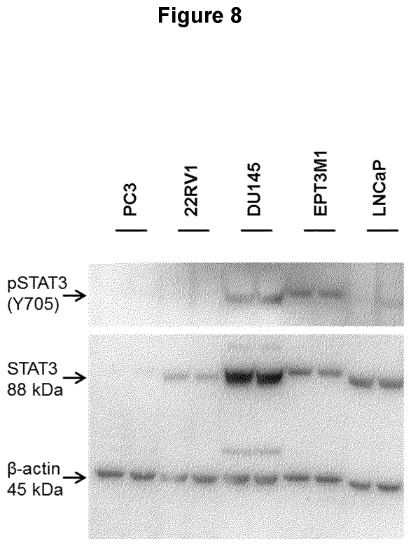

The expression of STAT3 and STAT1 in different cell lines was tested, and it was found that three cell lines (LNCaP, DU145, EPT3-M1-STAT3) expressed high basal levels of STAT3, whereas three cell lines (PC3, DU145, EPT3-M1-STAT3) showed abundant basal STAT1 expression (see FIGS. 6 and 7). Among these cell lines, IL-6-treatment for 15 minutes induced phosphorylation of STAT3 on Tyr705 only in LNCaP cells but not in DU145 or EPT3-M1-STAT3 cells, without affecting total STAT3 (see FIG. 8).

To obtain the results shown in FIG. 6, cell lysates were examined using Western blotting with specific antibodies.

To obtain the results shown in FIG. 7, various cell lines were treated with 10 ng/ml IL-6 for 15 min and lysates were examined using Western blotting with specific antibodies.

To obtain the results shown in FIG. 8, LNCaP cells were treated with DMSO, 20 μM 323-1 or 323-2 or 5 μM cryptotanshinone for 24 hrs before treatment with or without 10 ng/ml IL-6 for the last 15 min

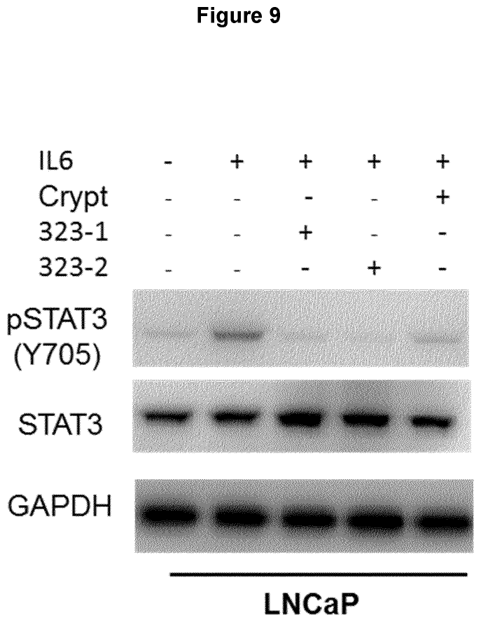

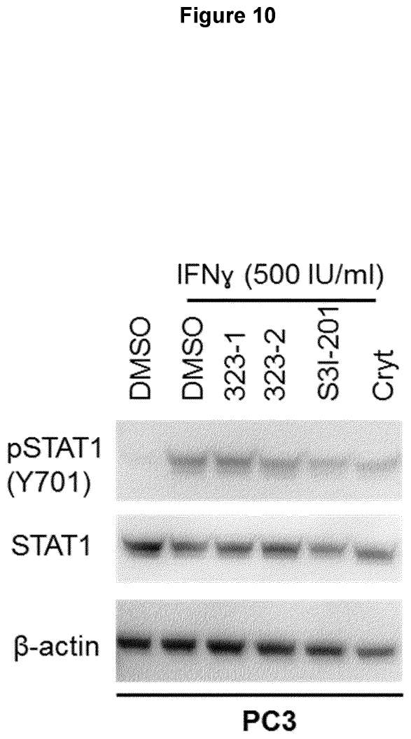

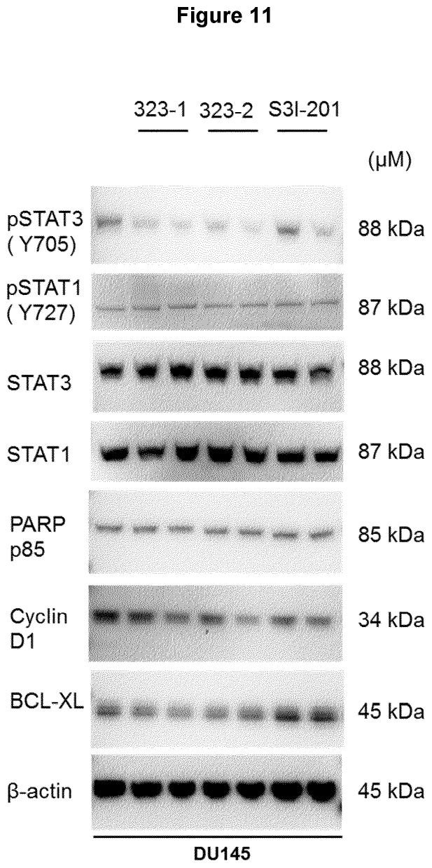

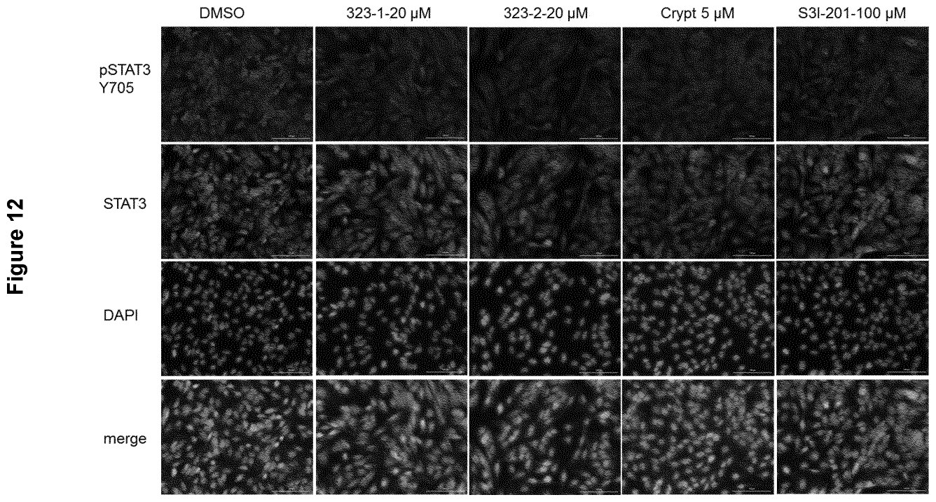

It was found that 323-1 and 323-2 inhibited IL-6-induced phosphorylation of STAT3 on Tyr705 in LNCaP cells (see FIG. 9), but not phosphorylation of STAT1 on Tyr-701 with IFN-γ in PC3 cells (see FIG. 10). It is reported that phosphorylated STAT1 (S727) can promote AML cells proliferation, accompanied by JAK-STAT pathway activation. Notably, 323-1 and 323-2 harbor the selective inhibitory effect of phosphorylated STAT3 (Tyr705) over pSTAT1 (S727) (see FIG. 11). As shown in FIG. 11, 323-1 and 323-2 impaired the expression of phosphorylated STAT3 at Tyr705 in various STAT3 high cells, especially with a better effect than S3I-201 in DU145 cells. This was further confirmed by performing an immunofluorescence assay in STAT3 highly expressed EPT3M1 cells with fusion GFP-STAT3 (see FIG. 12). Taken together, these findings suggest that 323-1 and 323-2 are small-molecule inhibitors of STAT3.

To obtain the results shown in FIG. 9, LNCaP cells were treated with DMSO, 20 μM of compound 323-1 or 323-2 or 5 μM cryptotanshinone for 24 hrs before treatment with or without 10 ng/ml IL-6 for the last 15 min.

To obtain the results shown in FIG. 10, PC3 cells were treated with DMSO, 20 μM of compound 323-1, 20 μM of compound 323-2, 100 μM S3I-201 or 5 μM cryptotanshinone for 24 hrs before treatment with or without 500 IU/ml IFNγ for the last 15 min.

To obtain the results shown in FIG. 11, DU145 cells were treated with indicated doses of compound 323-1, compound 323-2 or S3I-201 for 24 hrs.

To obtain the results shown in FIG. 12, cells were treated with DMSO, 20 μM of compound 323-1, 20 μM of compound 323-2, 100 μM S3I-201 or 5 μM cryptotanshinone for 24 hrs

323-1 and 323-2 Reduced the Protein Level of STAT3 Target Gene MCL1

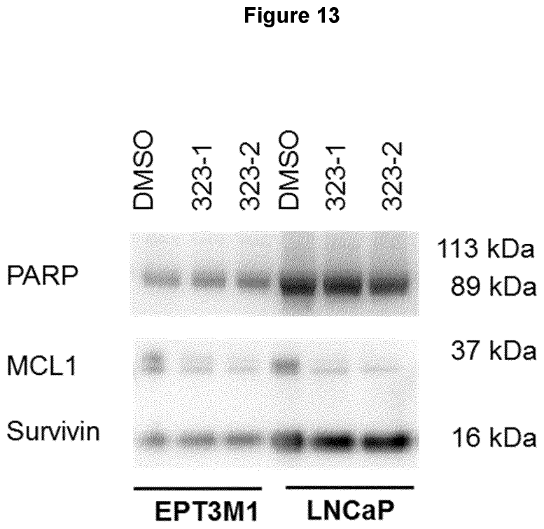

As reported, aberrant STAT3 regulates the expression of downstream target genes involved in anti-apoptosis (Bcl-2, Mcl-1, survivin, and Bcl-xL), cell cycle (cyclin D1 and c-Myc), angiogenesis (VEGF and HIF1α), invasion and metastasis (MMP-1, MMP-2, MMP-9) and the inhibition of host immune surveillance. To further identify the characterization of the properties of 323-1 and 323-2, DU145, EPT3-M1-STAT3 and LNCaP cells were treated with 323-1 and 323-2 followed by western blotting. FIG. 11 shows that the expression of Cyclin D1, but not BCL-XL in the LNCaP cells were down-regulated dose-dependently by these two compounds. Compound 323-1 and 323-2 repressed the protein level of STAT3 target gene MCL1 in both cell lines, but not Survivin or PARP in EPT3-M1-STAT3 and LNCaP cells (see FIG. 13). The results suggest that 323-1 and 323-2 modulate STAT3 target gene expression.

To obtain the results shown in FIG. 13, EPT3M1-STAT3 or LNCaP cells were treated with DMSO, 20 μM of compound 323-1, or 20 μM of compound 323-2 for 48 hrs, respectively. Lysates were analysed and loaded to western blotting.

323-1 and 323-2 Did not Affect the Binding of STAT3 to DNA

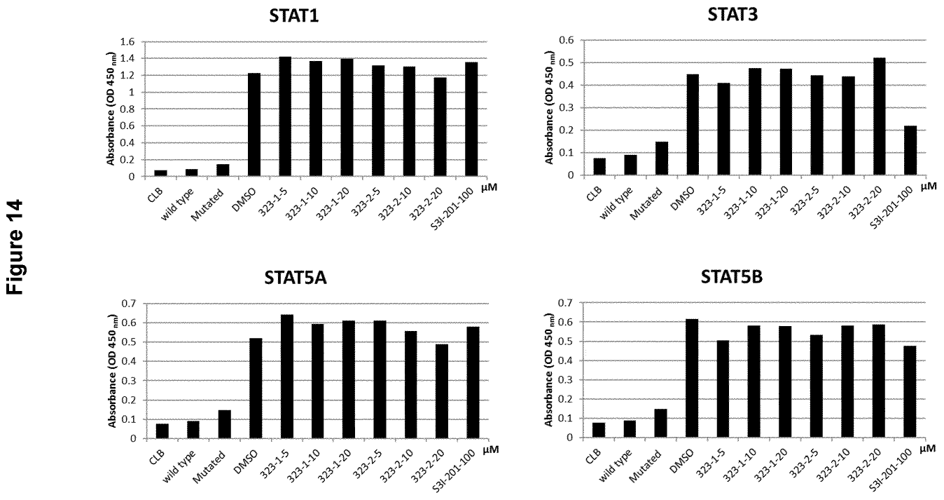

It is reported that many STAT3 inhibitors target the DBD of STAT3 or inhibit the binding of SH2 domain to pTyr705 residue for dimerization or to inhibit phosphorylation of Tyr705 for activation. Due to high sequence similarity within the highly conserved region of the SH2 domain compared with other proteins, such as Src, and insufficient capability of the current popular STAT3 SH2 domain inhibitors to suppress the aberrant STAT3 pathway, the interest for targeting STAT3 DBD is growing. To determine if compounds 323-1 and 323-2 targets the STAT3 DBD, nuclear lysates were exacted from the cells treated with drugs for 24 hrs and incubated with oligonucleotide containing STAT consensus binding site, following by adding the relevant primary (STAT1, STAT3, STAT5a or STAT5b) antibody. In contrast to the STAT3 DBD inhibitor S3I-201, neither 323-1 nor 323-2 affected the DNA-binding domain of STAT1, STAT3, STAT5A, STAT5B (see FIG. 14).

To obtain the results shown in FIG. 14, DU145 cells were seeded and treated with 5-20 μM of compound 323-1 or compound 323-2 for 24 hrs. Then nuclear proteins were extracted from the cell lysate for the DNA binding detection to STAT1, STAT3 and STAT5 according to the protocol as described in the Materials and Method. Complete Lysis Buffer (CLB) was set as blank. Nb2 nuclear extract (Prolactin stimulated) was added with Wild-type oligonucleotide (wild-type, competes with sample nuclear extracts for STAT) or the mutated oligonucleotide (mutated, no effect on ability of sample nuclear extracts to bind to STAT) into the pre-coated 96-well with STAT oligonucleotide as a control to monitor the specificity of the assay.

323-1 and 323-2 Targeted STAT3 Directly and Disrupted the STAT3 Dimerization

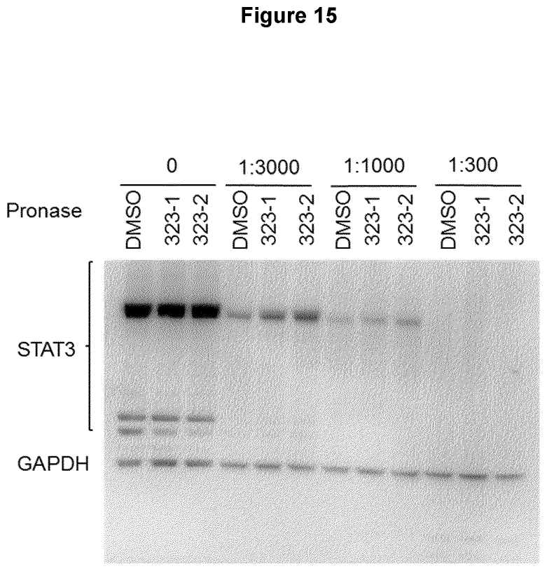

To directly address if 323-1 and 323-2 target STAT3 protein, drug affinity responsive target stability (DARTS) assay was used for target identification which is based on that the binding of targeted protein and drugs will be more stable against proteases. In FIGS. 15-18, Western blotting showed protection of the target protein STAT3, whereas digestion of the non-target proteins STAT1, JAK2, Src, and β-actin were unchanged by 323-1 and 323-2. This suggests that compounds 323-1 and 323-2 target STAT3 directly.

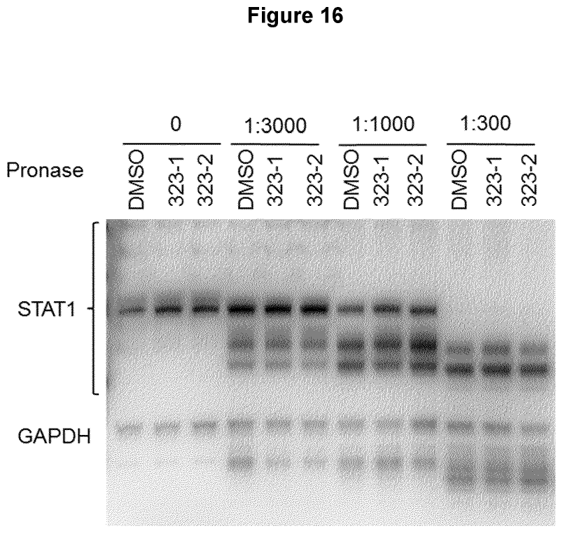

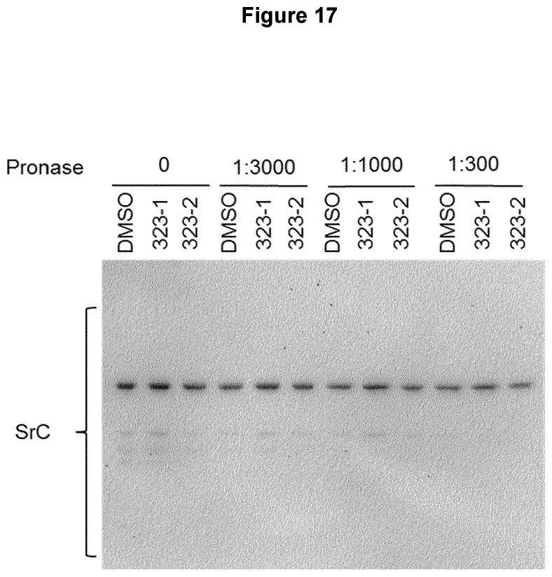

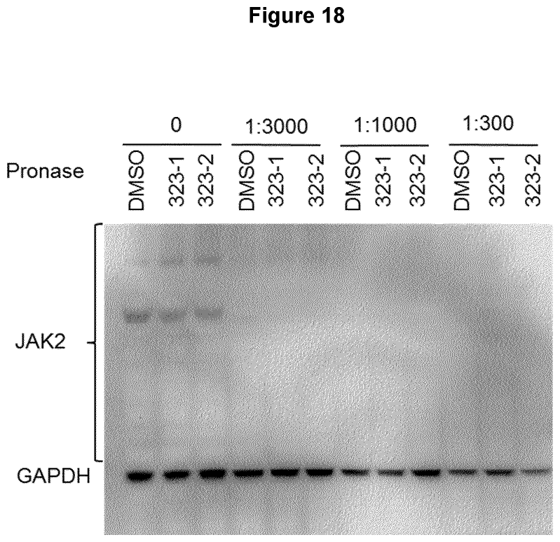

The EPT3M1-STAT3 cell lysates were prepared as described by Lomenick et al. (Proc Natl Acad Sci USA. 2009; 106: 21984-9), incubated with 200 μM 323-1 or 200 μM 323-1 or an equivalent amount of vehicle (DMSO) for 1 hr, followed by digestion with 1.25 mg/ml pronase (1:100 stock solution) at dilution ratios of 1:3000, 1:1000 and 1:300 by using 1×TNC buffer to create serial pronase stock aliquots for 1 hour. Then the samples were subjected to western blotting with antibodies against STAT3 (FIG. 15, ab119352, 1:2500), STAT1 (FIG. 16, ab119352), Src (FIG. 17), JAK2 (FIG. 18).

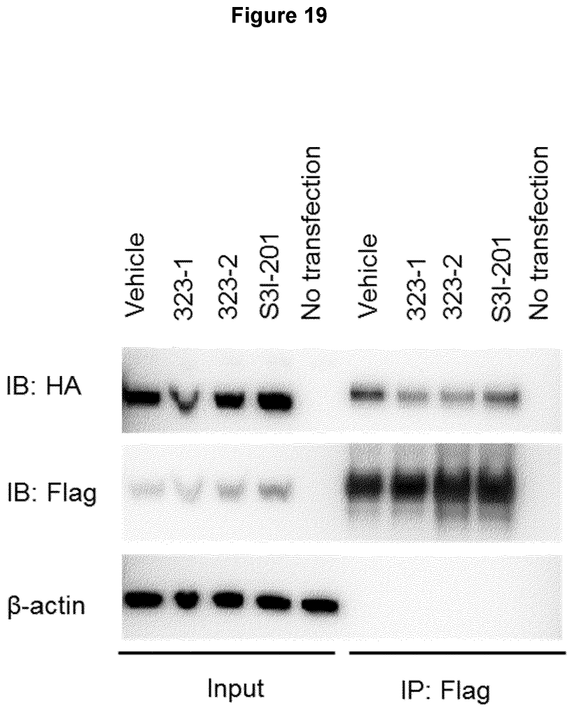

Nuclear translocation of the STAT3 dimers mediates the STAT3 transcriptional activity. In order to test whether compounds 323-1 and 323-2 are STAT3 dimerization inhibitors, 293T cells were transiently transfected with HA-tagged STAT3 and FLAG-tagged STAT3 plasmids, then stimulated with compounds 323-1 (50 μM) and 323-2 (50 μM) or S3I-201 (200 μM) for 24 hours, and then immunoprecipitated with anti-FLAG antibody to pull down the STAT3 associated proteins, which were then subjected to the Western blot and incubation with anti-HA, anti-Flag or anti-β actin.antibody. As shown in FIG. 19, it was confirmed that compounds 323-1 and 323-2 could disrupt the dimerization of STAT3 compared with S3I-201.

The Molecular Docking Data Supported that 323-1 and 323-2 Target the SH2 Domain of STAT3

The STAT3 SH2 domain (residues 583-688) contains three sub-pockets: PY, PY+1, and PY+X. The STAT3 SH2 domain facilitates the phosphorylation and dimerization of STAT3, due to its association between STAT3 monomers and phospho-tyrosine motifs within relevant receptors. To date, the STAT3 SH2 domain has become a dominating therapeutic target for small molecule modulator discovery and development. Several new STAT3 SH2 domain binding inhibitors have been identified through in silico computational screening assay by docking of compounds into the STAT3 SH2 domain, such as S3I-201.

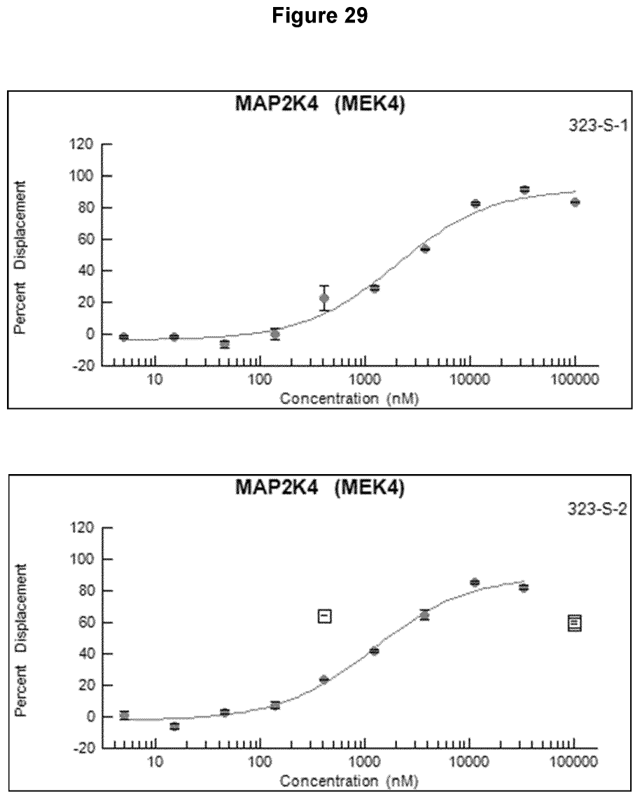

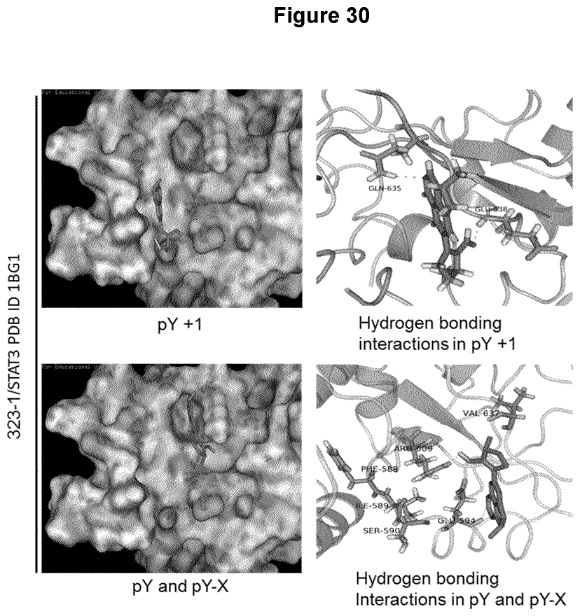

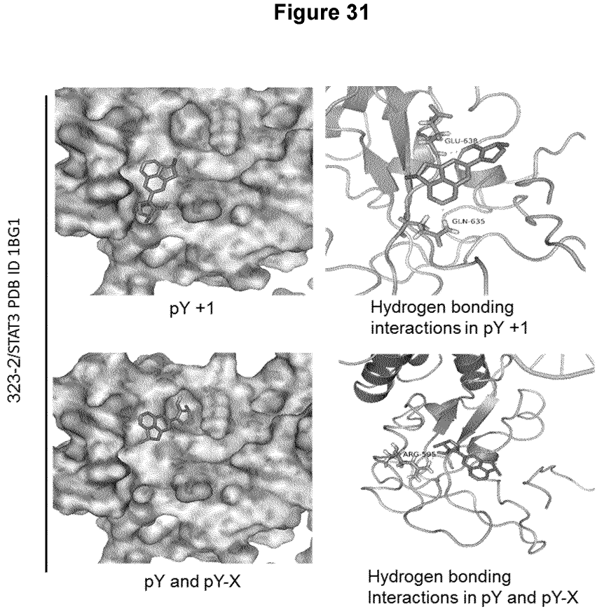

To confirm that compounds 323-1 and 323-2 bind to STAT3, in silico computational modelling and the Fluorescence Polarization (FP) Assay were used. Computational docking and molecular dynamics simulation identified potential binding sites for compounds 323-1 and 323-2 in the STAT3 SH2D (see FIGS. 30-32). In the molecular docking with phosphorylated (PDB entry 1BG1) STAT3, both compounds appeared to have potential binding ability to three pockets of STAT3 SH2 domain, PY, PY+1, and PY−X, whereas S3I-201 appeared able to bind only to the PY and PY−X pocket, not the PY+1 pocket. As shown in FIG. 31, the nitrogen of the isoquinoline group in compound 323-1 forms a hydrogen bond with Gln635, the carbonyl oxygen on the formaldehyde group forms a hydrogen bond with Glu638. The pY+1 pocket is mainly a hydrophobic region, and the small molecule skeleton cyclopenta[de]isoquinoline is facing this pocket, forming a hydrophobic interaction with surrounding amino acid residues (Ile634, Ser636, Val637). The nitrogen of the quinoline group in compound 323-1 forms a hydrogen bond with Glu 594, the carbonyl oxygen on the formaldehyde group faces the pY binding pocket and forms a hydrogen bond with Arg609, and Arg609 can form a direct polarity with phosphotyrosine 705. The role of Arg609 can inhibit the binding of SH2 to phosphotyrosine 705. The small molecule skeleton cyclopenta[de]isoquinoline faces the pY−X binding pocket and interacts hydrophobically with adjacent amino acid residues (Phe588, Ile589, Ser590, Val637). Similarly, compound 323-2 form a bond with PY, PY+1 and PY-X pocket (Gln635, Glu638, Ile634, Ser636, Val 637, Arg 595, Lys 591) in the STAT3 SH2 domain (see FIG. 30).

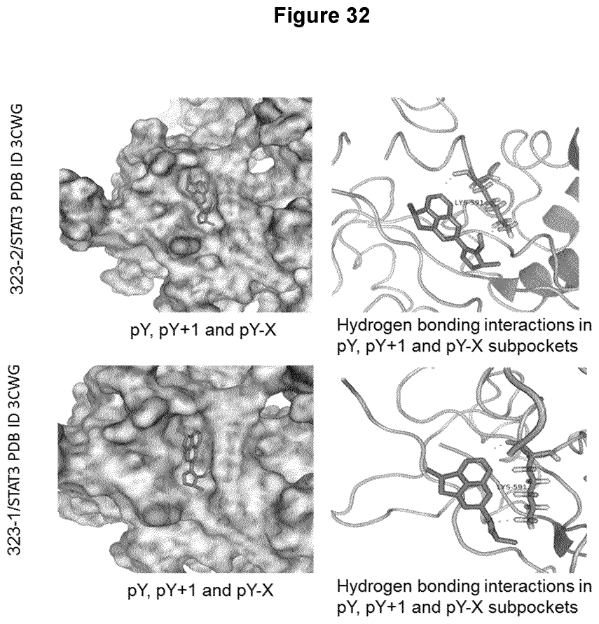

It is reported that the unphosphorylated STAT3 may also dimerize, enter the nucleus from the cytoplasm to bind to DNA and regulate as a transcriptional activator. In the docking with nonphosphorylated (PDB entry 3CWG) STAT3 structure, both of compounds 323-1 and 323-2 were predicted to directly interact with Tyr(P)-binding subpocket via hydrogen bonding within residues Lys-591 (see FIG. 32). Also, the cyclopenta[de]isoquinoline of compounds 323-1 and 323-2 associated with pY-X binding pocket bound by tetrahydro cyclopentane, and formed hydrophobic interactions lined by residues Phe588, Ile589, Ser590, Val637. Above all, these data show that compounds 323-1 and 323-2 are STAT3 SH2 domain inhibitors by disrupting the STAT3 dimerization, further supporting the results of DARTS assay.

323-1 and 323-2 Displaced the Binding of STAT3 to Fluorescein-Labelled pTyr-Peptide GpYLPQTV-NH2

The in silico predictions were further confirmed by competition binding FP assays, which detected the displacement by drugs of the binding of labelled phosphopeptide, 5-FLU-G(P-TYR)LPQTV-NH2 to the ligand human recombinant STAT3 protein. The phosphotyrosine peptide GpYLPQTV corresponds to the residues 903-909 within the gp-130 subunit of the IL-6 receptor, which has been validated to bind the STAT3-SH2 domain. Strategies to target STAT3 SH2 have been pursued recently. These small molecule inhibitors are designed to bind to a site resident in the STAT3 SH2 domain by competing with the p-Tyr705.

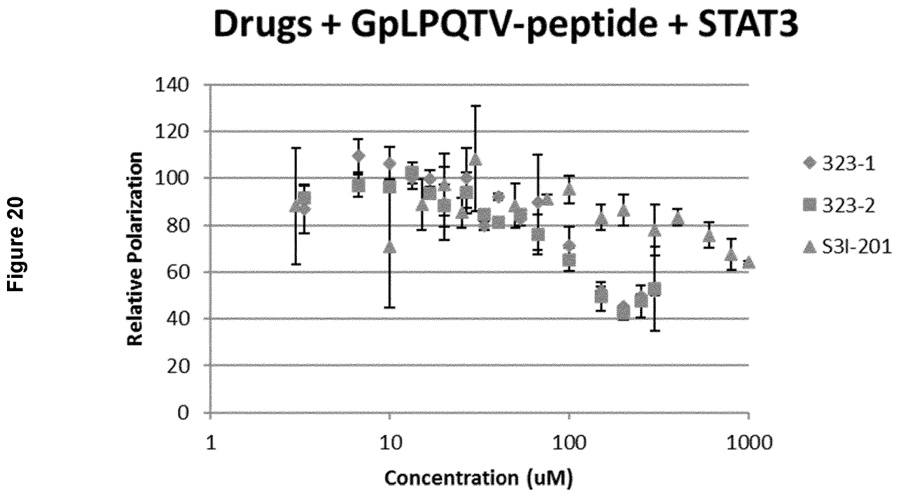

As STAT3 may be recruited to gp130 within phosphotyrosine residues, the competitive FP assay was utilized to identify whether 323-1 and 323-2 target STAT3 SH2 domain. FIG. 20 shows Fluorescence Polarization (FP) analysis of the binding of 10 nM GpYLTQTV-NH₂ and 150 nM human STAT3 protein as well as an increasing concentration of drugs. FIG. 20 shows that 323-1 and 323-2 disrupted the binding of STAT3 to the phosphotyrosine peptide GpYLPQTV with Ki values of around 94 μM and 75 μM, respectively.

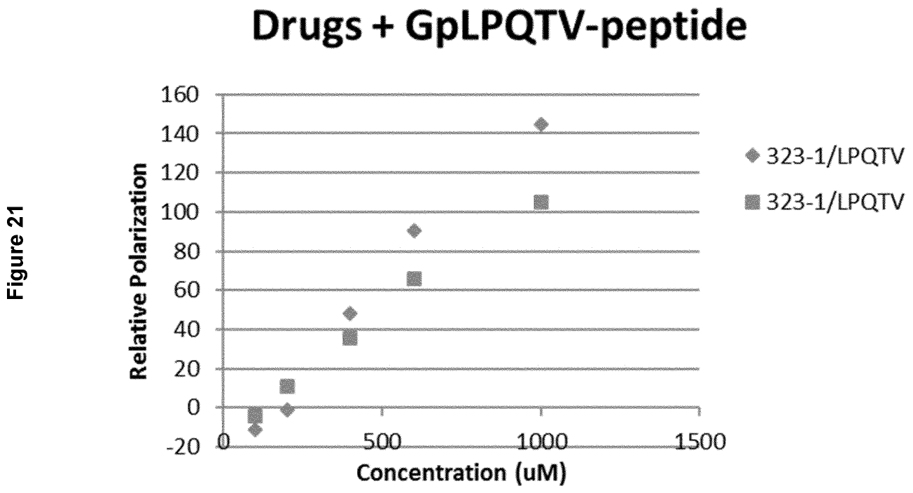

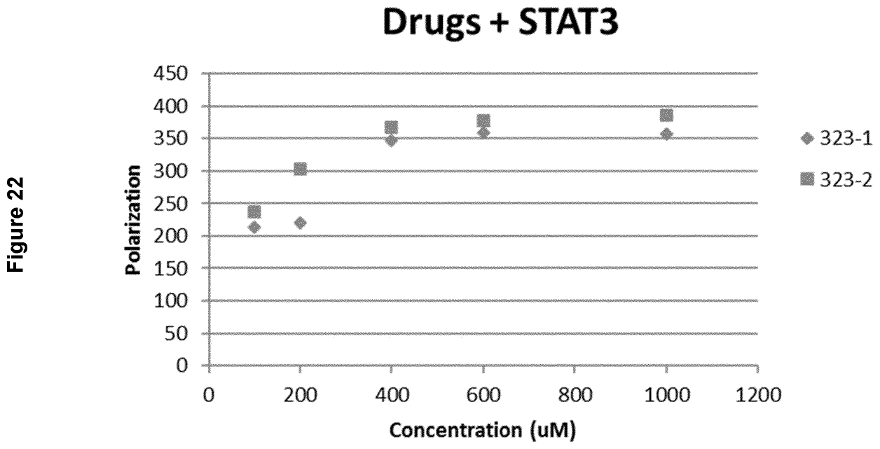

The potency of compounds 323-1 and 323-2 to disrupt the STAT3/GpYLPQTV was around 5 fold higher than the S3I-201 Kd values of around 529 μM. Notably, both 323-1 and 323-2 have an increase in polarization at concentrations above 200 μM. These compounds do not appear to be fluorescent at the measured wavelengths, so at high concentrations some interaction may be occurring between drug and fluorophore to decrease the fluorophore's mobility due to their autofluorescence. This was checked by measuring the polarization of peptide in the presence of high concentrations of the drug and without STAT3 (see FIGS. 21 and 22). Different doses of drugs were combined with 10 nM GpYLTQTV-NH₂ (FIG. 21) or 150 nM human STAT3 protein (FIG. 22) to check if the autofluorescence of drugs affects the final signal.

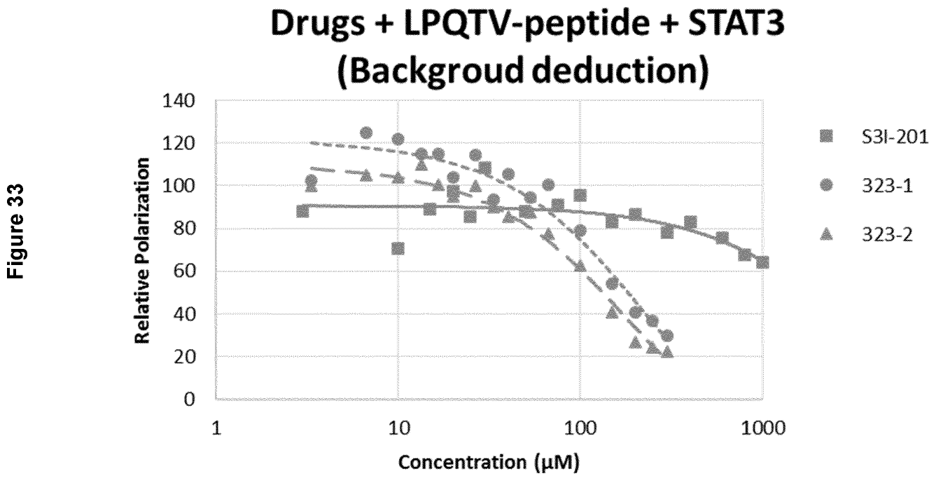

In order to subtract the background and minimize the interference of the autofluorescence, administration with only peptides and ligands was used to validate the efficacy of protein-ligand interaction with Ki values of 94 μM for 323-1 and 75 μM for 323-2 (see FIG. 33).

FIG. 33 shows Fluorescence Polarization analysis of background deduction by application of 10 nM GpYLTQTV-NH2 and increasing doses of drugs. The control was set as 1% DMSO. Data are representative of 2 independent experiments.

Taken together, these data confirm that 323-1 and 323-2 are direct STAT3 SH2 inhibitors by disrupting the binding of STAT3 phosphotyrosine-peptide, which is consistent with the computational docking and DARTS assay.

323-1 and 323-2 Decreased the Ability to Form Colonies and Induced Apoptosis

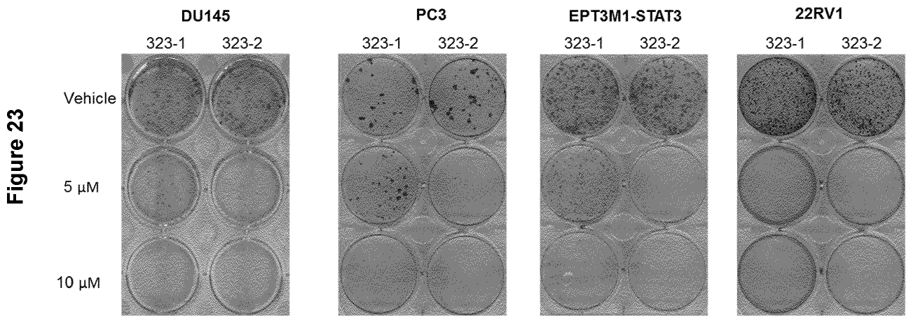

The effect of 323-1 and 323-2 concerning the inhibition of clonogenicity was investigated in vitro clonogenic assays, which correspond very well to tumorigenicity in nude mice. On the second day after seeding single cells, 323-1 and 323-2 were added at various concentrations, and cells were allowed to grow for 2 weeks to form colonies, then stained with 0.4% crystal violet (w/v). Concentration-dependent inhibition of colony formation in four cancer lines, DU145, PC3, EPT3M1-STAT3, and 22Rv1 cells, was notably observed after exposure to 323-1 and 323-2 (see FIG. 23).

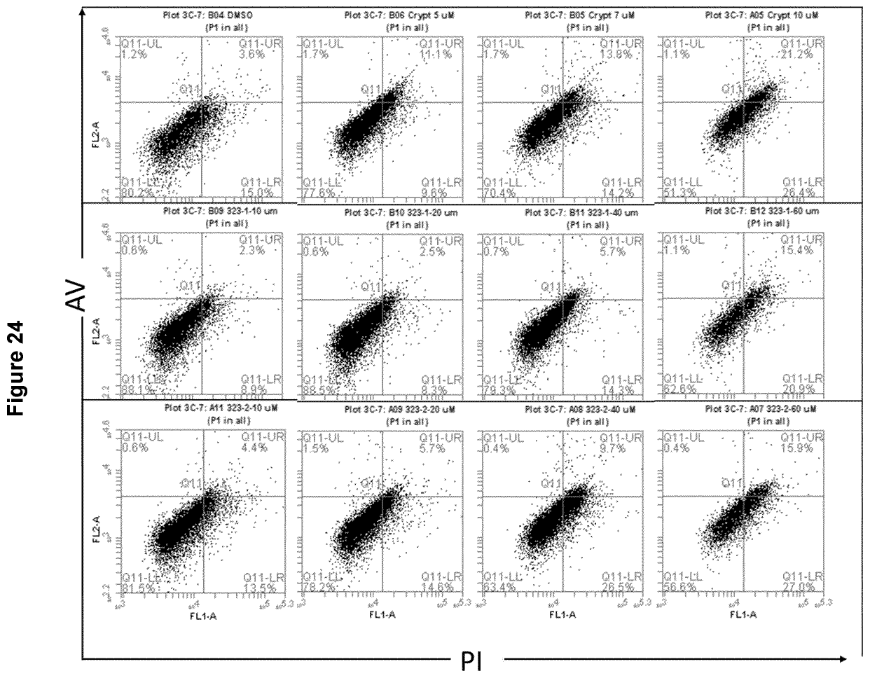

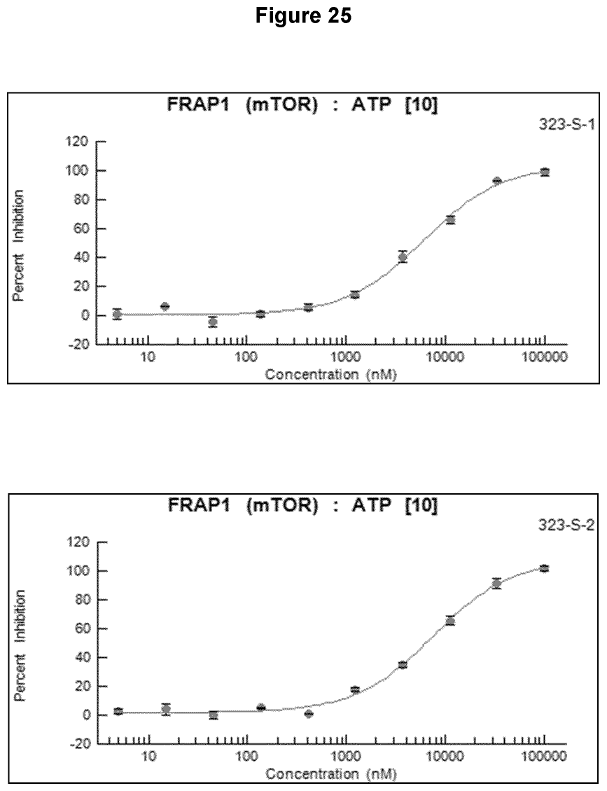

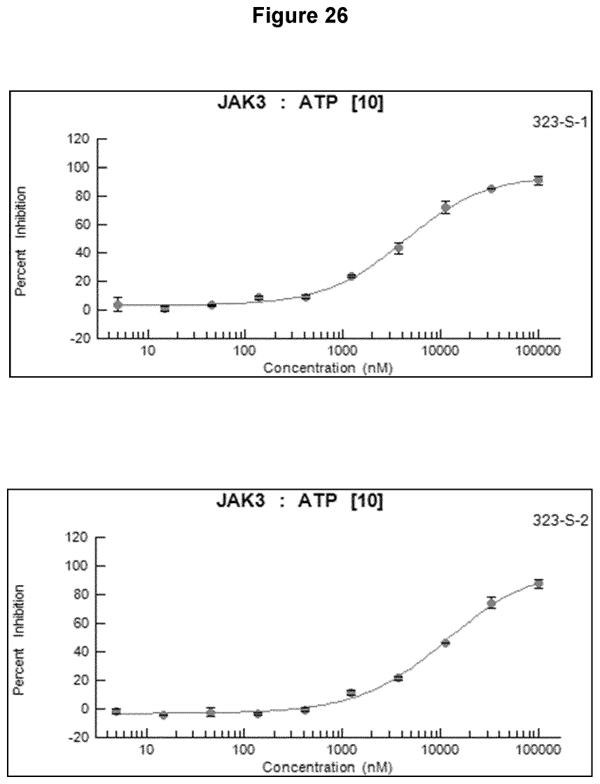

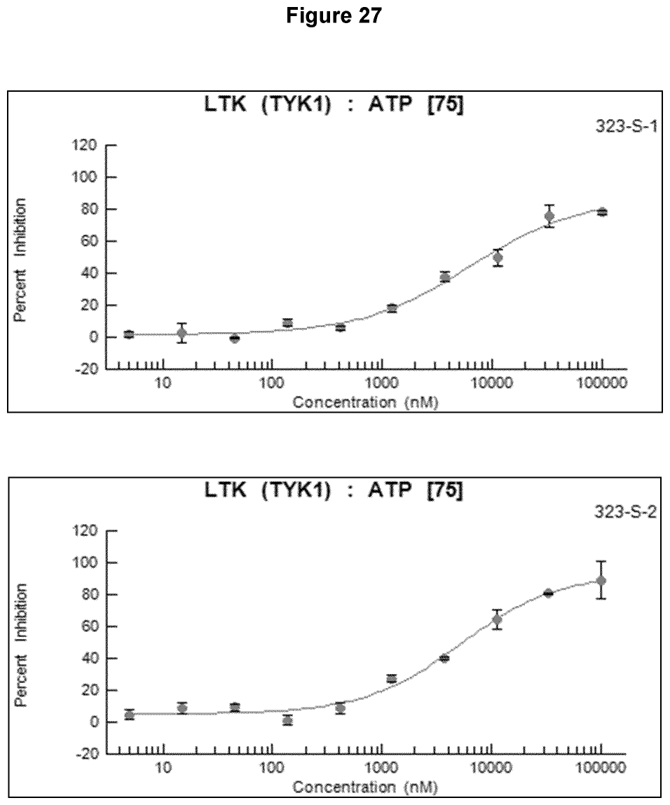

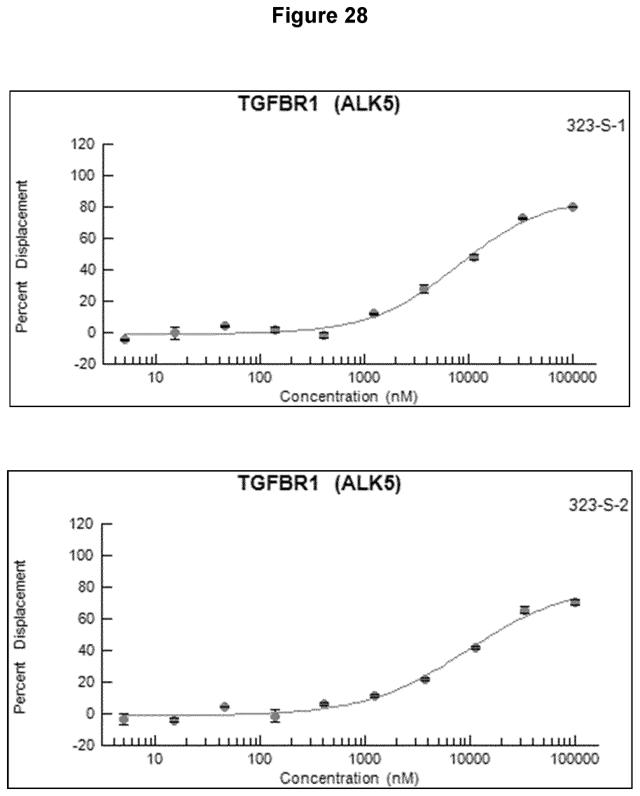

The anti-apoptotic effect of compounds 323-1 and 323-2 was evaluated in DU145 cells. As shown in FIG. 24, 323-1 and 323-2 induced apoptosis-like changes in many cells and appeared to induce apoptosis in DU145 cells treated for 72 hrs. By contrast, induction of apoptosis by compounds 323-1 and 323-2 was much weaker than by cryptotanshinone, which is reported to possess high cytotoxic effect.

FIG. 24 shows flow cytometric analysis of cell apoptosis induced by treatment with 323-1, 323-2 or cryptotanshinone for 72 hrs in DU145 cells. Data are shown as representative results of three experiments.

To sum up, all these results indicate that compounds 323-1 and 323-2 possess in vitro anti-tumour activity by inducing apoptosis and inhibiting cell proliferation and clonogenicity.

Compound 323s Regressed Development of CRPC Tumors

To further test the effect of compounds 323-1 and 323-2 against prostate cancer, the human prostate cancer cell line LNCaP (which expresses full-length androgen receptor) was injected into a NOD/SCID mouse, to establish a humanised NOD/SCID mouse CRPC model. A total of 5×10⁶ cells were subcutaneously injected into the right flank of BALB/c-nu nude mice.

The mice were treated daily for three weeks with IP injections of either vehicle (olive oil) or compound 323-2 (20 mg/kg or 40 mg/kg in the vehicle). Body weights and tumour volumes were measured before each drug injection. After the 22nd day, the mice were sacrificed, and the tumours were collected, weighed and stored at −70° C.

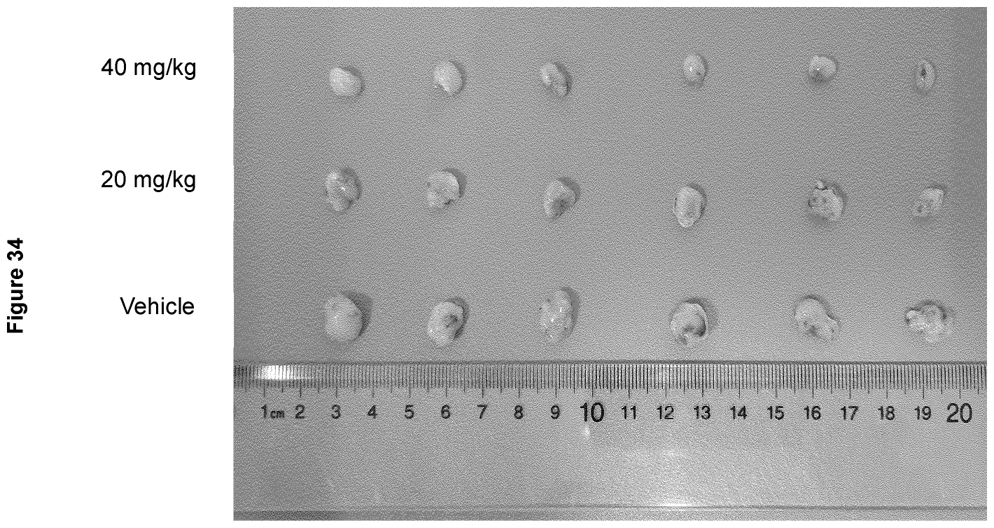

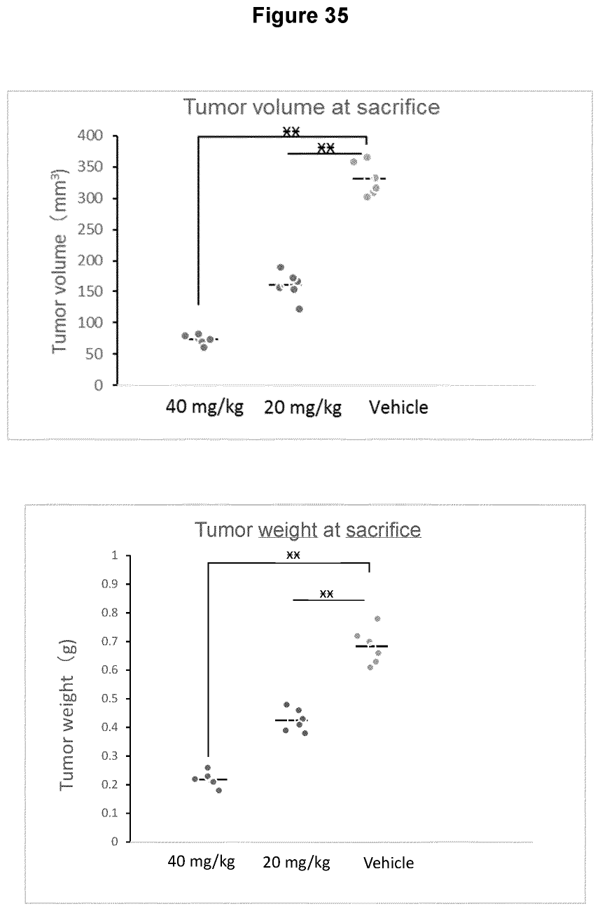

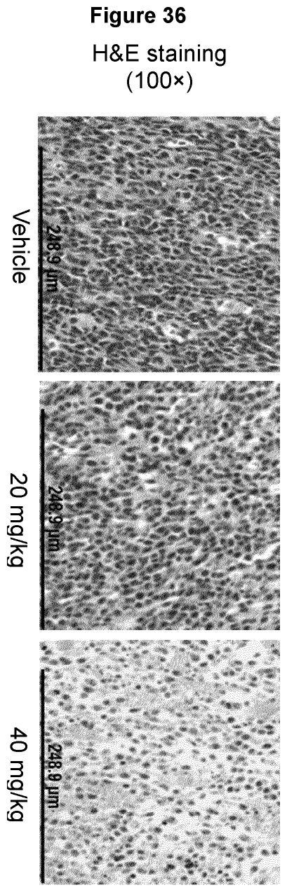

FIG. 34 shows a photograph of the harvested LNCaP tumours from each group on the day of sacrifice. FIG. 35 shows the individual animal tumour volume and weight on the day of sacrifice in each group. FIG. 36 shows H&E staining of harvested representative LNCaP tumours in each group.

These data show that compound 323-2 significantly inhibited CRPC tumour growth, especially for the high dose group (40 mg/kg). No significant body weight change was observed and no mice died during the whole drug administration.

Work Related to Immunotherapy

Low platelet peripheral blood mononuclear cells (PBMCs) were isolated from peripheral blood of healthy donors by Ficoll-Paque density centrifugation. Monocytes were derived from the above PBMCs by immunomagnetic selection with CD14+ microbeads (Pan Monocyte Isolation Kit from Miltenyi Biotec) and seeded at s 0.5×106 monocytes/ml in CellGro medium supplemented with GM-CSF (100 ng/ml) and IL-4 (20 ng/ml) to obtain immature dendritic cells (iDCs). Cytokines were replenished after 72 hrs and 24 hrs after incubation, iDCs were stimulated with 30 ng/ml LPS to obtain mature dendritic cells (moDCs). Test compounds were added into the wells 1 hr prior to the LPS treatment and co-incubated with LPS for another 23 hrs. Supernatant was collected for the detection of secreted IL-10 and IL-12p70 using a standard ELISA kit and cells were collected and markers identified by flow cytometry.

Compounds 323-1 and 323-2 Triggered Immune-Suppression in Dendritic Cells

The effects of compounds 323-1 and 323-2 and various controls on the LPS-induced expression of cytokines IL-10, IL-12p70, co-stimulatory molecules and maturation markers (CD80, CD83, CD86, HLA-DR, CCR7) and programmed death-ligand 1 (PD-L1) and programmed death-ligand 2 (PD-L2) were determined.

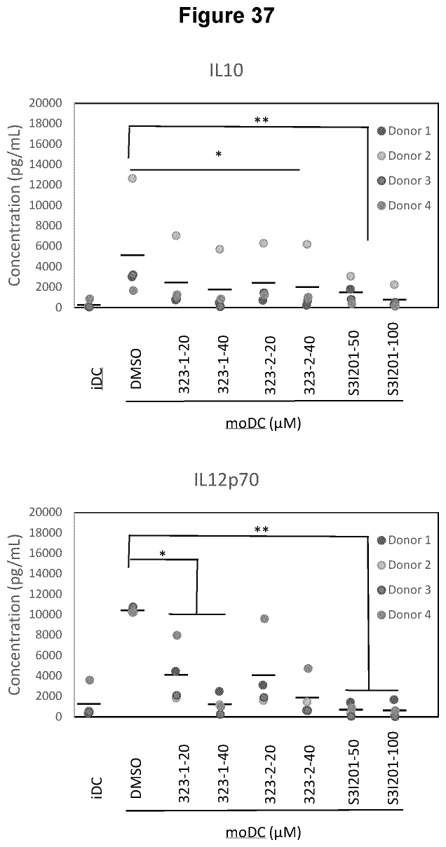

After treatment with compound 323-1 or 323-2 for 24 hrs, both compounds significantly reduced the secretion of IL-10 and IL-12p70 (FIG. 37).

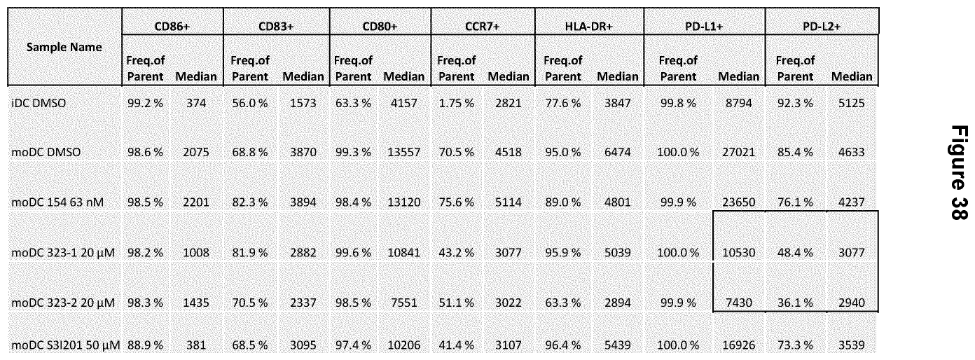

Notably, 323s clearly inhibited the LPS-induced dendritic markers (CD80, CD83, CD86, HLA-DR, CCR7). Interestingly, a similar immune-suppression effect was also observed for the expression of PD-L1 and PD-L2. The results of this flow cytometry analysis are presented in the table in FIG. 38. The first two rows are controls and there are two positive controls in compounds 154 and S-31201. S-31201 is a known commercial STAT3 inhibitor, 154 has been published as a dual AR/STAT3 inhibitor. Compared with these two STAT3 inhibitors, the 323s are much more potent as PD-L1/L2 modulators.

Current immune check point inhibitors of PD-L1 have been proven as an effective approach in cancer immunotherapy. Thus, compounds 323-1 and 323-2 also have potential in STAT3i checkpoint blockade immunology.

323-1 and 323-2 are Kinase Inhibitor of JAK3, TYK1, TGFB1, MAP2K4 and FRAP1

The activation of STAT3 upon tyrosine phosphorylation can be triggered by the cytokine receptor-associated kinases such as JAKs, certain growth factor receptor tyrosine kinases (RTKs) such as EGF, FGF, PDGF and VEGF, and the non-receptor-associated tyrosine kinases like Src. The selectivity of 323-1 and 323-2 was examined by the kinases profiling against 36 key kinases in Table 1.

TABLE 1
Enzayme Inhibitory Activity of 323-
S-1 and 323-S on Various Kinases

| | | IC₅₀ (nM) | |
|---|---|---|---|
| Kinase | Classification | 323-1 | 323-2 |
| JAK3 | Cytoplasmic Tyr | 4260 | 11600 |
| LTK (TYK1) | Receptor Tyr | 6220 | 5250 |
| FRAP1 (mTOR) | Ser/Thr | 6130 | 7520 |
| TGFBR1 (ALK5) | Ser/Thr | 7950 | 9250 |
| MAP2K4 (MEK4) | Ser/Thr | 2010 | 1220 |
| AKT1 (PKB alpha) | | >20000 | >20000 |
| AKT2 (PKB beta) | | >20000 | >20000 |
| EGFR (ErbB1) | | >20000 | >20000 |
| ERBB2 (HER2) | | >20000 | >20000 |
| ERBB4 (HER4) | | >20000 | >20000 |
| FGFR1 | | >20000 | >20000 |
| FGFR2 | | >20000 | >20000 |
| FGFR3 | | >20000 | >20000 |
| FGFR4 | | >20000 | >20000 |
| FLT1 (VEGFR1) | | >20000 | >20000 |
| FLT4 (VEGFR3) | | >20000 | >20000 |
| HCK | | >20000 | >20000 |
| JAK1 | | >20000 | >20000 |
| JAK2 | | >20000 | >20000 |
| KDR (VEGFR2) | | >20000 | >20000 |
| LYN A | | >20000 | >20000 |
| LYN B | | >20000 | >20000 |
| MAP2K6 (MKK6) | | >20000 | >20000 |
| MAPK1 (ERK2) | | >20000 | >20000 |
| MAPK10 (JNK3) | | >20000 | >20000 |
| MAPK3 (ERK1) | | >20000 | >20000 |
| MAPK8 (JNK1) | | >20000 | >20000 |
| MAPK9 (JNK2) | | >20000 | >20000 |
| MET (cMet) | | >20000 | >20000 |
| PDGFRA (PDGFR alpha) | | >20000 | >20000 |
| PDGFRB (PDGFR beta) | | >20000 | >20000 |
| PRKCD (PKC delta) | | >20000 | >20000 |
| SRC N1 | | >20000 | >20000 |
| SRC | | >20000 | >20000 |
| TYK2 | | >20000 | >20000 |
| TGFBR2 | | >20000 | >20000 |

Among the 36 kinases, it was found that both compounds 323-1 and 323-2 inhibit these kinases, JAK3, TYK1, TGFB1, MAP2K4 and FRAP1 (see FIGS. 25-29), which contributed to the STAT3 activation. In cell free assays, the IC50 of 323-1 for enzyme activity of JAK3 was 4260 nM, TYK1 (6220 nM), FRAP1 (6130 nM), TGFBR1 (7950 nM), MAP2K4 (2010), and the IC50 of 323-2 for enzyme activity of was JAK3 (11600 nM), TYK1 (5250 nM), FRAP1 (7520 nM) TGFBR1 (9250 nM), MAP2K4 (1220). The integration of the simultaneous inhibitory effect of 323-1 and 323-2 on these five kinases and STAT3 SH2 domain is hypothesized to result in the efficient STAT3 inactivation, especially in STAT3-driven tumors.

Inhibition by compounds 323-1 and 323-2 of MAP2K4 (MEK4) and TGFBR1 (ALK5) was determined by Z′-LYTE™ Screening. Inhibition by compounds 323-1 and 323-2 of FRAP1 (mTOR), JAK3, and LTK (TYK1) was determined by LanthaScreen Eu Kinase Binding Assay Service using the Adapta Universal Kinase Assay (Life Technologies). The 1% DMSO was used as the negative control.

### Example 2—Methods of Synthesis

Salts of Compounds 323-1 and 323-2

Dissolve a 27.7 g (0.1 mol) sample of compound 323-1 or 323-2 and 17.7 g (0.1 mol) fumaric acid (or 0.1 mol of another organic or inorganic acid) in 200 ml ethanol, and then heat in a water bath until all samples are dissolved. Filter the solution using diatomaceous earth, cool the filtrate slowly, and stand for 5 h at 0-5° C. The crystals can be precipitated, filtrated and dried to give the fumarate (or other) salts of compound 323-1 or 323-2.

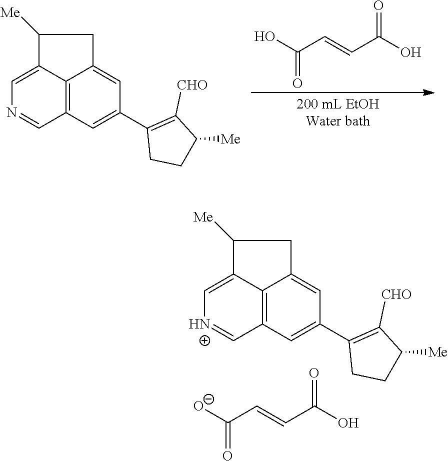 [SMILES: CC1Cc2cc(C3=C(C=O)[C@H](C)CC3)cc3cncc1c23 ; O=C(O)/C=C/C(=O)O ; O=C([O-])/C=C/C(=O)O ; CC1Cc2cc(C3=C(C=O)[C@H](C)CC3)cc3c[nH+]cc1c23]

Alcohol and Ester Derivatives of Compounds 323-1 and 323-2

Under the conditions of 0° C. and nitrogen protection, add 27.7 mg (0.1 mmol) of compound 323-1 or 323-2, 5 mL THF and 57 mg (1.5 mmol) LiAlH₄ to a 25 mL double-necked flask, and then react under stirring for 1 h at room temperature. Add 2 mL 16% NaOH solution to the reaction system for quenching reaction. Wash the reaction mixture with ethyl acetate (7 mL×3). Wash the combined organic phase with citric acid, saturated NaCl solution in turn, and dry with anhydrous Na₂SO₄. Obtain the crude product (compound I as shown below) by filtration and concentration, by column chromatography eluting with petroleum ether (60-90):ethyl acetate=3:1(volume ratio).

Under the condition of 0° C. and nitrogen protection, add 27.9 mg (0.1 mmol) of compound I, 10 mL THF carboxylic acid (0.12 mmol), DCC (1.5 mmol) and DMAP (0.5 mmol) to a 25 mL double-necked flask, and then react under stirring for 24 h at room temperature. Remove dicyclohexyl urea by filtration, and wash the filtrate twice with 0.5 mol/L hydrochloric acid, followed twice with saturated aqueous sodium bicarbonate solution. Dry the organic phase over anhydrous magnesium sulfate, and evaporate the solvent in vacuo to give a white solid, then submit over silica gel column chromatography eluting with ethyl acetate:petroleum ether=2: 1 to yield solid product compound 11-1.

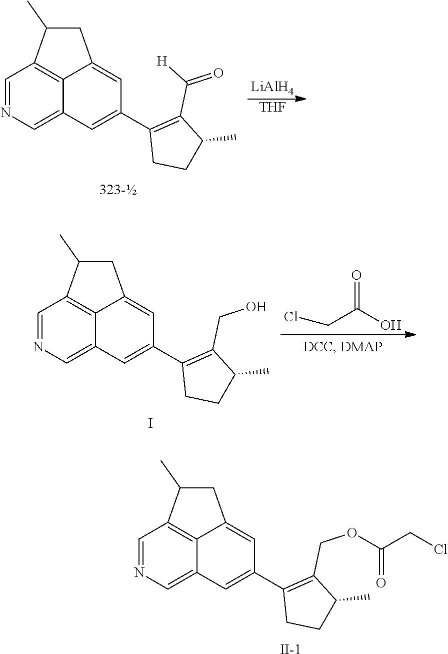

Carboxylic Acid and Ester Derivatives of Compounds 323-1 and 323-2

Under the condition of 0° C. and nitrogen protection, add 27.7 mg (0.1 mmol) of compound 323-1 or 323-2, 2 mL H₂O, 5 mL BtOH and NaH₂PO₄ (1.5 mmol) to a 25 mL double-necked flask, and then react under stirring for 1 h at room temperature. Add a solution of sodium chlorite (NaClO₂, 2.0 mmol) to the reaction system, and stir for 2 h. Add sodium bisulfite to the reaction system for quenching reaction. Wash the reaction mixture with ethyl acetate (10 mL×3). Dry the combined organic phase with anhydrous Na₂SO₄. The crude product (compound III as shown below) may be obtained by filtration and concentrated, which can be purified by silica gel column chromatography eluting with petroleum ether (60-90):ethyl acetate=3:1(volume ratio).

Under the condition of 0° C. and nitrogen protection, add 29.3 mg (0.1 mmol) of compound Ill, 10 mL THF alcohol (0.12 mmol), DCC (1.5 mmol) and DMAP (0.5 mmol) to a 25 mL double-necked flask, and then react under stirring for 24 h at room temperature. Remove dicyclohexyl urea by filtration. Wash the filtrate twice with 0.5 mol/L hydrochloric acid, and then twice with saturated aqueous sodium bicarbonate solution. Dry the organic phase over anhydrous magnesium sulfate, and evaporate the solvent in vacuo to obtain a white solid, which may be chromatographed over silica gel eluting with ethyl acetate:petroleum ether=2: 1 to give solid product IV-1.

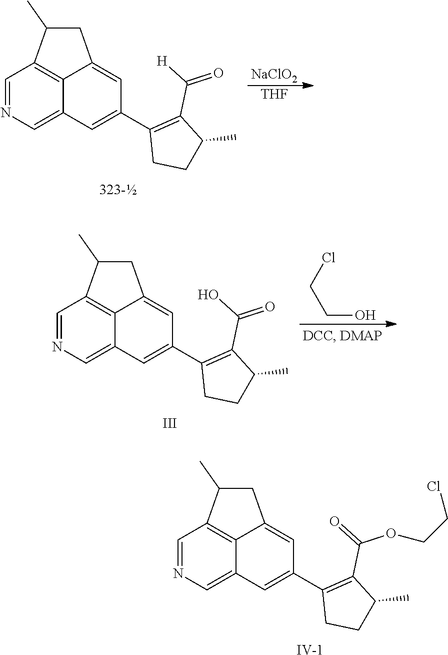

Acetal Derivatives of Compounds 323-1 and 323-2

Add 27.7 mg (0.1 mmol) of compound 323-1 or 323-2, anhydrous methanol (2.0 mL), and a catalytic amount of PPTS (0.008 g, 0.032 mmol) to a 25 mL double-necked flask at 0° C. and under nitrogen protection. Stir the reaction mixture for 1 hour, and then dilute with CH₂Cl₂ and treat with a saturated NaHCO₃ solution. Extract the aqueous layer with CH₂Cl₂ (2×15 mL). Dry the combined organic layer with anhydrous Na₂SO₄, filter, and concentrate in vacuo. Purification by flash chromatography will give compound V-1 as shown below.

Under the condition of 0° C. and nitrogen protection, add 32.3 mg (0.1 mmol) of compound V-1, 5 mL CH₃OH and NaBH₄ (1.5 mmol) to a 25 mL of double-necked flask, and then react under stirring for 1 h at room temperature. Add 10 mL of a saturated NaCl solution and ethyl acetate to the reaction system for quenching. Wash the reaction mixture with ethyl acetate (10 mL×3). Dry the combined organic phase with anhydrous Na₂SO₄. The crude product (compound V-3 as shown below) may be obtained by filtration and concentrated, and purified by column chromatography with petroleum ether (60-90):ethyl acetate=3:1(volume ratio).

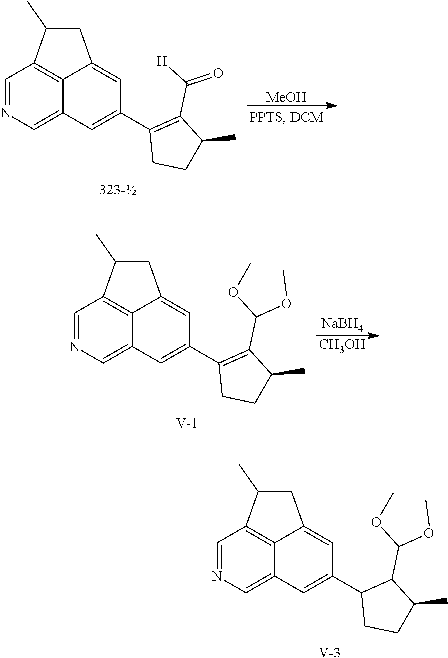

Halogenated Derivatives of Compounds 323-1 and 323-2

To an oven-dried round-bottom flask charged with compound 323-1 or 323-2 (277 mg, 1.0 mmol) in CH₂Cl₂, add mCPBA (77% w/w, 11 mmol) portionwise over 15 min at room temperature. Stir the reaction mixture for 16 h at room temperature. Add PPh₃ (5 mmol) to reduce any unreacted peracid, and stir the mixture for 2 h at room temperature. Evaporate the solvent under reduced pressure. The residue may be purified by flash column chromatography (CH₂Cl₂/MeOH=30:1) to afford the corresponding azine-N-oxides.

Load a reaction vial with azine-N-oxidesxx (586 mg, 2.0 mmol), triethylamine (0.53 mL, 4.0 mmol) in dibromomethane (3 mL). Cool the resulting solution to approximately −60° C., then add a solution of oxalyl bromide (0.57 mL, 4.0 mmol) dropwise. After 0.5 h, quench the reaction mixture with the addition of MeOH (0.5 mL). Warm the reaction to room temperature. Wash the dicloromethane layer with saturated NH₄Cl (3.0 mL) and water (3.0 mL); and dry and concentrate the organic layer with MgSO₄ to give a crude product. Purify the crude product by silica gel chromatography, eluted with 50% dichloromethane in heptane. Pure fractions may be collected and evaporated to give product 1.

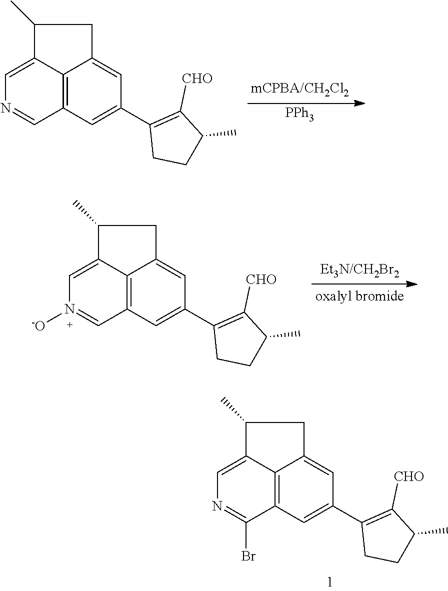

To a reaction tube equipped with a magnetic stir bar, add compound 323-1 or 323-3 (69.29 mg, 0.25 mmol), sodium bromide (127 mg, 1.25 mmol), potassium persulfate (135 mg, 0.5 mmol), and 1.0 mL of dichloromethane (DCM). Stir the mixture at room temperature in a closed tube. Monitor the reaction by TLC. The product may be extracted with EtOAc and the combined organic layers dried over Na₂SO₄, and concentrated under vacuum. The crude mixture may be purified by silica gel column chromatography using 15% EtOAc/hexane to afford product 2.

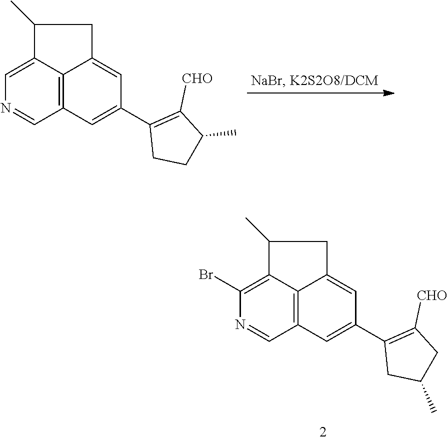 [SMILES: CC1Cc2cc(C3=C(C=O)[C@H](C)CC3)cc3cncc1c23 ; CC1Cc2cc(C3=C(C=O)C[C@H](C)C3)cc3cnc(Br)c1c23]

Oxidative Derivatives of Compounds 323-1 and 323-2

Stir a solution of compound 323-1 or 323-2 (277 mg, 1 mmol), TBHP (1.14 ml, 10 mmol), and Ebselen (63.5 mg, 0.25 mmol) in t-butyl alcohol (5 ml) at 80° C. until the substrate is exhausted. After 30 h, filtrate the yellow solid and add a pinch of Pd=asbestos (10%) to decompose peroxides, and evaporated the solvent in vacuo. The crude solid residue may be purified by column chromatography on silica gel (hexane/EtOAc=4/1), to give pure compound 3 as shown below.

To a solution of compound 3 (291 mg, 1 mmol) in MeOH (4.2 mL) and CH₂Cl₂ (4.2 mL) at 0° C., add NaBH₄ (95.1 mg, 2.51 mmol). Stir the reaction mixture for 30 min at room temperature, and then quench the reaction with saturated aqueous NH₄Cl at 0° C. Separate the organic layer, and extract the aqueous layer with EtOAc. Dry the combined organic layer over Na₂SO₄. After filtration and evaporation of the solvent, the crude product may be purified by column chromatography on silica (hexane/EtOAc=4/1) to afford compound 4 as shown below.

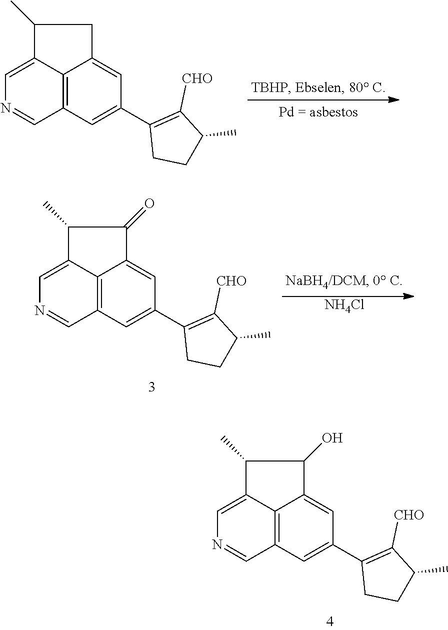

Amide Derivatives of Compounds 323-1 and 323-2

Add diphenyl phosphoryl azide (1.2 equiv) to a solution of an alcohol derivative of compound 323-1 or 323-2 (279 mg) in dry toluene ([alcohol]=0.7-1.2 M) under argon. Cool the reaction mixture at 0° C., stir for 10 min, and add DBU (1.2 equiv) slowly over 20 min. Stir the mixture for 2 h at 0° C., then for 20 h at r.t., and remove the solvent in vacuo. Dissolve the residue in a 1:1 EtOAc/hexane mixture, filter through a short silica pad, concentrate, dissolve in dry THF ([azide]=0.1 M), and add Ph₃P (1.2 equiv) and H₂O (2 equiv). After refluxing overnight, the reaction mixture may be diluted with H₂O and extracted with CH₂Cl₂. Purification by flash chromatography can afford the corresponding primary amine compound 323-1 or 323-2 (as shown in the reaction scheme below).

To a stirred mixture of carboxylic acid (7 mmol) in anhydrous dichloromethane (20 mL) add dropwise a dichloromethane (15 mL) solution of dicyclohexylcarbodiimide (7 mmol) at 0° C. followed by a dichloromethane (15 mL) solution of the primary amine of compound 323-1 or 323-2 (279 mg, 1 mmol). Stir the mixture at 0° C. for 30 min and then warm to room temperature for another 2 h. After the reaction is complete, add 0.5 mL of acetic acid, and stir the mixture for an additional 30 min. Remove insoluble dicyclohexylurea by filtration. After removing the solvent, the residue may be dissolved in diethyl ether and then filtered again. Remove the excessive carboxylic acid though washing with saturated NaHCO₃ aqueous (2×30 mL) and brine (2×30 mL), and dry the organic phase over anhydrous Na₂SO₄. Evaporation of the organic solvent can afford the product the amide of compound 323-1 or 323-2 (compound 5 as shown below).

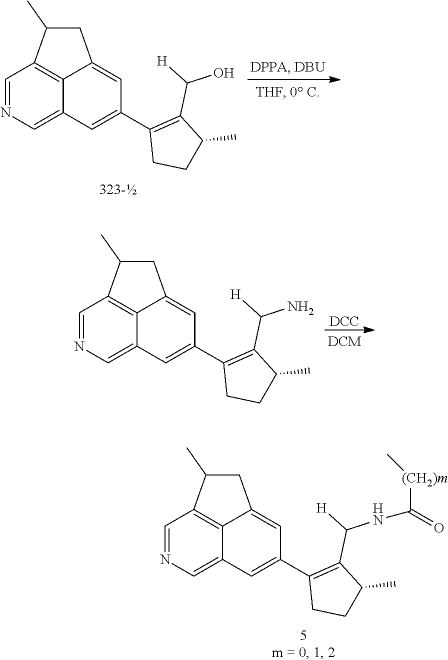

Amine Derivatives of Compounds 323-1 and 323-2

To a vented, 100 mL, two-neck flask equipped with a magnetic stirrer, add 1 mmol of the amide of compound 323-1 or 323-2, 0.8 g (20 mmol) of NaBH₄, and 20 mL of Me₂SO. Mix methane-sulfonic acid (1.7 ml, 27.5 mmol) and 10 ml of Me₂SO and add dropwise to the reaction mixture by means of an addition funnel over a 30-min period. During this addition, stir the reaction mixture constantly. When the reaction is complete, quench the reaction mixture by the addition of 20 ml of 10% NaOH. Extract the product from the reaction mixture with three 10-ml portions of methylene chloride. Wash the product solution with three 10-ml portions of 0.1 M NaOH to remove most residual Me₂SO, and extract the product into three 10-ml portions of 10% HCl. Neutralize with 10% NaOH, followed by extraction with three 10-ml portions of methylene chloride, drying over Na₂SO₄. Evaporation of the organic solvent can afford the product the amine derivatives of 323-1/2 (compound 6 as shown below).

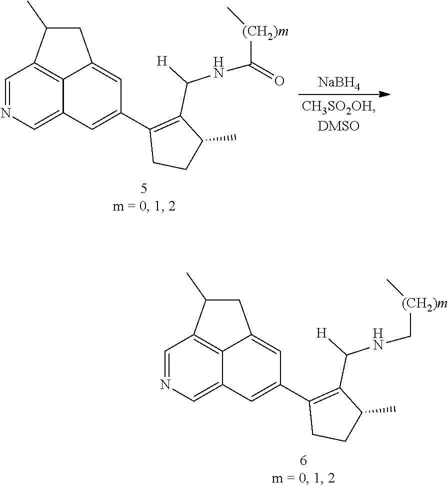

1. A method of treating cancer selected from prostate, stomach, small intestine, oesophagus, melanoma, head or neck, kidney, bladder, urinary, brain, lung, pancreas, endometrium, thyroid, bile duct, gall bladder, blood vessel, appendix and rectum cancer; or selected from Chronic Lymphocytic Leukaemia and Acute Lymphocytic Leukaemia, wherein the method comprises administering to a patient in need thereof a therapeutically effective amount of a compound of Formula (I), or a solvate, a tautomer, a stereoisomer or a salt thereof; 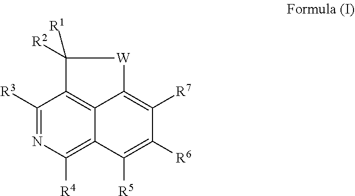
wherein
W is selected from the group consisting of —CH₂—, —CHOH—, and —C(═O)—;
R¹, R², R³, R⁴, R⁵, and R⁷ are each independently selected from the group consisting of —H, —OH, a halogen, and C₁₋₃ alkyl;
R⁶ is selected from the group consisting of C₅₋₇ cycloalkyl substituted by R^{x} and R^{y}, and C₅₋₇ cycloalkenyl substituted by R^{x} and R^{y};
R^{x} is selected from the group consisting of —OH, —NH₂, C₁₋₆ aldehyde, C₁₋₆ alcohol, C₁₋₆ alkoxy, C₂₋₆ ether, C₁₋₆ carboxylic acid, C₂₋₆ ester, C₁₋₆ amine, C₁₋₆ amide, C₂₋₆ hemiacetal, C₃₋₆ acetal, and C₃₋₄ cyclic acetal, each of which may be substituted by one or more R^{a} group;
R^{a} is selected from the group consisting of —OH, a halogen, and —NH₂; and
R^{y} is selected from the group consisting of —H, —OH, a halogen, and C₁₋₃ alkyl.

2. The method according to claim 1, wherein W is —CH₂—.

3. The method according to claim 1, wherein R¹ is —H and R² is Me.

4. The method according to claim 1, wherein R³ and R⁴ are each independently selected from the group consisting of —H and a halogen.

5. The method according to claim 1, wherein R⁵ and R⁷ are both —H.

6. The method according to claim 1, wherein R⁶ is C₅₋₆ cycloalkenyl, which is substituted by R^{x} and R^{y} or wherein R⁶ is 1-cyclopentenyl, which is substituted by R^{x} and R^{y}.

7. (canceled)

8. The method according to claim 1, wherein R^{x} is selected from the group consisting of C₁₋₆ aldehyde, C₂₋₆ hemiacetal, C₃₋₆ acetal, and C₃₋₄ cyclic acetal, each of which may be substituted by one or more R^{a} group, or wherein R^{x} is —CHO, or wherein R^{y} is C₁₋₃ alkyl.

9. (canceled)

10. (canceled)

11. The method according to claim 1, wherein the compound is a compound of Formula (II): 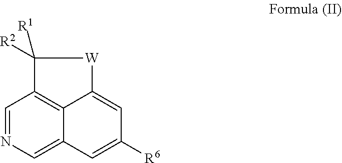
Wherein R¹ and R² are each independently selected from the group consisting of —H, —OH, a halogen, and C₁₋₃ alkyl, and wherein R⁶ is selected from the group consisting of C₅₋₇ cycloalkyl substituted by R^{x} and R^{y}, and C₅₋₇ cycloalkenyl substituted by R^{x} and R^{y}, wherein R^{x} is selected from the group consisting of —OH, —NH₂, C₁₋₆ aldehyde, C₁₋₆ alcohol, C₁₋₆ alkoxy, C₂₋₆ ether, C₁₋₆ carboxylic acid, C₂₋₆ ester, C₁₋₆ amine, C₁₋₆ amide, C₂₋₆ hemiacetal, C₃₋₆ acetal, and C₃₋₄ cyclic acetal, each of which may be substituted by one or more R^{a} group, wherein R^{a} is selected from the group consisting of —OH, a halogen, and —NH₂, and wherein R^{y} is selected from the group consisting of —H, —OH, a halogen, and C₁₋₃ alkyl.

12. The method according to claim 1, wherein the compound is a compound of Formula (III): 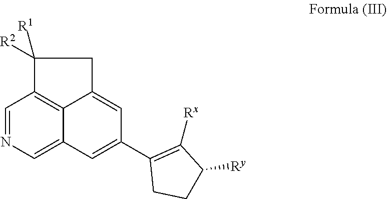
Wherein R¹ and R² are each independently selected from the group consisting of —H, —OH, a halogen, and C₁₋₃ alkyl, and wherein R^{x} is selected from the group consisting of —OH, —NH₂, C₁₋₆ aldehyde, C₁₋₆ alcohol, C₁₋₆ alkoxy, C₂₋₆ ether, C₁₋₆ carboxylic acid, C₂₋₆ ester, C₁₋₆ amine, C₁₋₆ amide, C₂₋₆ hemiacetal, C₃₋₆ acetal, and C₃₋₄ cyclic acetal, each of which may be substituted by one or more R^{a} group, wherein R^{a} is selected from the group consisting of —OH, a halogen, and —NH₂, and wherein R^{y} is selected from the group consisting of —H, —OH, a halogen, and C₁₋₃ alkyl.

13. The method according to claim 1, wherein the compound is a compound of Formula (IV): 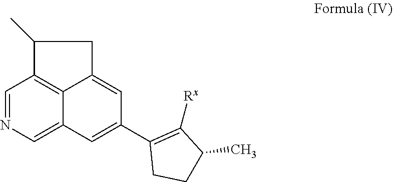
wherein R^{x} is selected from the group consisting of —OH, —NH₂, C₁₋₆ aldehyde, C₁₋₆ alcohol, C₁₋₆ alkoxy, C₂₋₆ ether, C₁₋₆ carboxylic acid, C₂₋₆ ester, C₁₋₆ amine, C₁₋₆ amide, C₂₋₆ hemiacetal, C₃₋₆ acetal, and C₃₋₄ cyclic acetal, each of which may be substituted by one or more R^{a} group, wherein R^{a} is selected from the group consisting of —OH, a halogen, and —NH₂.

14. The method according to claim 1, wherein the method is a method of treating prostate cancer.

15. The method according to claim 14, wherein the prostate cancer is castration resistant prostate cancer.

16. The method according to claim 14, wherein the prostate cancer is metastatic prostate cancer.

17. A compound of Formula (I) or a solvate, a tautomer, a stereoisomer or a salt thereof: 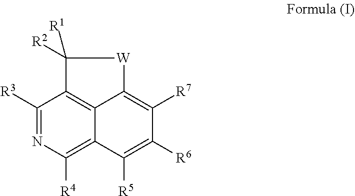
with the proviso that the compound is not:

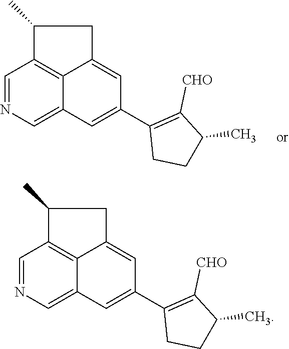 [SMILES: C[C@@H]1CCC(c2cc3c4c(cncc4c2)[C@H](C)C3)=C1C=O ; C[C@@H]1CCC(c2cc3c4c(cncc4c2)[C@@H](C)C3)=C1C=O]

18. A composition comprising a compound as defined in claim 14 and a pharmaceutically acceptable carrier.

19. (canceled)

20. (canceled)

21. (canceled)

22. A method of inhibiting STAT3 or the STAT3 pathway in a patient, wherein the method comprises administering to said patient a therapeutically effective amount of a compound according to claim 1.

23. The method of claim 22, wherein the method is a method of inhibiting the STAT3 SH2 domain.

24. A method of cancer immunotherapy, wherein the method comprises comprises administering to a patient in need thereof a therapeutically effective amount of a compound as defined in claim 1.

25. A method according to claim 24, wherein the compound is used in combination with an immune checkpoint inhibitor and/or a tumour microenvironment modulator.

26. A pharmaceutical pack or pharmaceutical composition comprising:
(i) a compound as defined in claim 1; and
(ii) an immune checkpoint inhibitor and/or a tumour microenvironment modulator.

27. The method according to claim 24 or 25 wherein the cancer is selected from the group consisting of prostate, stomach, small intestine, oesophagus, melanoma, head, neck, kidney, bladder, urinary, brain, lung, pancreas, endometrium, thyroid, bile duct, gall bladder, blood vessel, appendix and rectum cancer, Chronic Lymphocytic Leukaemia and Acute Lymphocytic Leukaemia, head and neck squamous cell carcinoma, non-small cell lung cancer (squamous and non-squamous carcinoma), melanoma, urothelial cancer, Merkel cell carcinoma, refractory Hodgkin lymphoma, microsatellite instability-high colorectal cancer and gastric cancer.
